(19) United States
(12) Patent Application Publication
(10) Pub. No.: US 2023/0072281 A1
(43) Pub. Date: Mar. 9, 2023

(54) Methods and Systems for Engineering Wavelet-Based Features From Biophysical Signals for Use in Characterizing Physiological Systems

(71) Applicant: Analytics for Life Inc., Toronto (CA)

(72) Inventors: Farhad Fathieh, North York (CA); Timothy William Fawcett Burton, Ottawa (CA)

(21) Appl. No.: 17/891,259

(22) Filed: Aug. 19, 2022

Related U.S. Application Data
(60) Provisional application No. 63/235,968, filed on Aug. 23, 2021.

(51) Int. Cl. A61B 5/00; A61B 5/024; A61B 5/318; G16H 50/20

(52) U.S. Cl. CPC A61B 5/7264; A61B 5/726; A61B 5/0059; A61B 5/02416; A61B 5/318; G16H 50/20

(57) ABSTRACT

The exemplified methods and systems facilitate the use for diagnostics, monitoring, treatment of one or more wavelet-based features or parameters determined from biophysical signals such as cardiac/biopotential signals and/or photoplethysmography signals that are acquired non-invasively. The wavelet-based features or parameters can be used, in one embodiment, within a model or classifier (e.g., a machine-learned classifier) to estimate metrics associated with the physiological state of a subject, including for the presence or non-presence of a disease or abnormal condition. Wavelet-based features or parameters may include measures that are derived from extractable properties or geometric characteristics of a spectral image or data of high-power spectral contents or high-coherence in waveform signals of interest in an acquired biophysical signal. Wavelet-based features or parameters may also include measures that are derived from a statistical quantification of the distribution of the power of the high-power spectral contents in the waveform signals of interest.

### RELATED APPLICATION

This US application claims priority to, and the benefit of, U.S. Provisional Patent Application No. 63/235,968, filed Aug. 23, 2021, entitled “Methods and Systems for Engineering Wavelet-Based Features From Biophysical Signals for Use in Characterizing Physiological Systems,” which is incorporated by reference herein in its entirety.

### FIELD OF THE INVENTIONS

The present disclosure generally relates to methods and systems for engineering features or parameters from biophysical signals for use in diagnostic applications; in particular, the engineering and use of wavelet-based features for use in characterizing one or more physiological systems and their associated functions, activities, and abnormalities. The features or parameters may also be used for monitoring or tracking, controls of medical equipment, or to guide the treatment of a disease, medical condition, or an indication of either.

### BACKGROUND

There are numerous methods and systems for assisting a healthcare professional in diagnosing disease. Some of these involve the use of invasive or minimally invasive techniques, radiation, exercise or stress, or pharmacological agents, sometimes in combination, with their attendant risks and other disadvantages.

Diastolic heart failure, a major cause of morbidity and mortality, is defined as symptoms of heart failure in a patient with preserved left ventricular function. It is characterized by a stiff left ventricle with decreased compliance and impaired relaxation leading to increased end-diastolic pressure in the left ventricle, which is measured through left heart catheterization. Current clinical standard of care for diagnosing pulmonary hypertension (PH), and for pulmonary arterial hypertension (PAH), in particular, involves a cardiac catheterization of the right side of the heart that directly measures the pressure in the pulmonary arteries. Coronary angiography is the current standard of care used to assess coronary arterial disease (CAD) as determined through the coronary lesions described by a treating physician. Non-invasive imaging systems such as magnetic resonance imaging and computed tomography require specialized facilities to acquire images of blood flow and arterial blockages of a patient that are reviewed by radiologists.

It is desirable to have a system that can assist healthcare professionals in the diagnosis of cardiac disease and various other diseases and conditions without the aforementioned disadvantages.

### SUMMARY

A clinical evaluation system and method are disclosed that facilitate the use of one or more wavelet-based features or parameters determined from biophysical signals such as cardiac/biopotential signals and/or photoplethysmography signals that are acquired, in preferred embodiments, non-invasively from surface sensors placed on a patient while the patient is at rest. The wavelet-based features or parameters can be used in a model or classifier (e.g., a machine-learned classifier) to estimate metrics associated with the physiological state of a patient, including for the presence or non-presence of a disease, medical condition, or an indication of either. The estimated metric may be used to assist a physician or other healthcare provider in diagnosing the presence or non-presence and/or severity and/or localization of diseases or conditions or in the treatment of said diseases or conditions.

The estimation or determined likelihood of the presence or non-presence of a disease, condition, or indication of either can supplant, augment, or replace other evaluation or measurement modalities for the assessment of a disease or medical condition. In some cases, a determination can take the form of a numerical score and related information.

Examples of wavelet-based features or parameters include measures that are derived from extractable properties or geometric characteristics of a spectral image or data of high-power spectral contents or high-coherence in waveform signals of interest in an acquired biophysical signal. Additional examples of wavelet-based features or parameters include measures that are derived from a statistical quantification of the distribution of the power of the high-power spectral contents in the waveform signals of interest.

As used herein, the term “feature” (in the context of machine learning and pattern recognition and as used herein) generally refers to an individual measurable property or characteristic of a phenomenon being observed. A feature is defined by analysis and may be determined in groups in combination with other features from a common model or analytical framework.

As used herein, “metric” refers to an estimation or likelihood of the presence, non-presence, severity, and/or localization (where applicable) of one or more diseases, conditions, or indication(s) of either, in a physiological system or systems. Notably, the exemplified methods and systems can be used in certain embodiments described herein to acquire biophysical signals and/or to otherwise collect data from a patient and to evaluate those signals and/or data in signal processing and classifier operations to evaluate for a disease, condition, or indicator of one that can supplant, augment, or replace other evaluation modalities via one or more metrics. In some cases, a metric can take the form of a numerical score and related information.

In the context of cardiovascular and respiratory systems, examples of diseases and conditions to which such metrics can relate include, for example: (i) heart failure (e.g., left-side or right-side heart failure; heart failure with preserved ejection fraction (HFpEF)), (ii) coronary artery disease (CAD), (iii) various forms of pulmonary hypertension (PH) including without limitation pulmonary arterial hypertension (PAH), (iv) abnormal left ventricular ejection fraction (LVEF), and various other diseases or conditions. An example indicator of certain forms of heart failure is the presence or non-presence of elevated or abnormal left-ventricular end-diastolic pressure (LVEDP). An example indicator of certain forms of pulmonary hypertension is the presence or non-presence of elevated or abnormal mean pulmonary arterial pressure (mPAP).

### BRIEF DESCRIPTION OF THE DRAWINGS

The accompanying drawings, which are incorporated in and constitute a part of this specification, illustrate embodiments and, together with the description, serve to explain the principles of the methods and systems.

Embodiments of the present invention may be better understood from the following detailed description when read in conjunction with the accompanying drawings. Such embodiments, which are for illustrative purposes only, depict novel and non-obvious aspects of the invention. The drawings include the following figures:

FIG. 1 is a schematic diagram of example modules, or components, configured to non-invasively compute wavelet-based features or parameters to generate one or more metrics associated with the physiological state of a patient in accordance with an illustrative embodiment.

FIG. 2 shows an example biophysical signal capture system or component and its use in non-invasively collecting biophysical signals of a patient in a clinical setting in accordance with an illustrative embodiment.

FIGS. 3A-3B each shows an example method to use wavelet-based features/parameters or their intermediate outputs in a practical application for diagnostics, treatment, monitoring, or tracking in accordance with an illustrative embodiment.

FIGS. 4A and 4B each illustrates an example wavelet feature computation module configured to determine values of wavelet associated properties of one or more acquired biophysical signals in accordance with an illustrative embodiment.

FIG. 5 illustrates an example wavelet distribution feature computation module configured to determine values of wavelet distribution associated properties of one or more acquired biophysical signals in accordance with an illustrative embodiment.

FIGS. 6A, 6B, and 6C show example methods, e.g., of the modules of FIGS. 4A, 4B, and 5, respectively, to generate wavelet-based features or parameters in accordance with an illustrative embodiment.

FIGS. 7A-7B each shows an example method of pre-processing cardiac signals and photoplethysmographic signals, respectively, in accordance with an illustrative embodiment.

FIGS. 8A-8F show example methods to delineate regions of spectral interest of cardiac signals, photoplethysmographic signals, and velocityplethysmogram signals for subsequent wavelet-based spectral analysis in accordance with an illustrative embodiment.

FIGS. 9A-9D show example methods of the wavelet feature module of FIG. 4A to generate wavelet-based features from a binarized spectral image of the spectral wavelet model of cardiac signals, photoplethysmographic signals, and velocityplethysmogram signals, in accordance with an illustrative embodiment.

FIGS. 10A-10F show example methods of the wavelet feature module of FIG. 4B to compute wavelet-based features from trends established in multiple binarized spectral images of the spectral wavelet model of cardiac signals, photoplethysmographic signals, and velocityplethysmogram signals, in accordance with an illustrative embodiment.

FIG. 11 shows example methods of the wavelet feature module of FIG. 4A to generate a wavelet-based feature from a coherence spectral image generated from multiple biophysical signals in accordance with an illustrative embodiment.

FIGS. 12A-12B, 13A-13D, and 14A-14D show example methods of the wavelet distribution feature module of FIG. 5 to generate wavelet distribution features in accordance with an illustrative embodiment.

FIG. 15A shows a schematic diagram of an example clinical evaluation system configured to use wavelet-based features among other computed features to generate one or more metrics associated with the physiological state of a patient in accordance with an illustrative embodiment.

FIG. 15B shows a schematic diagram of the operation of the example clinical evaluation system of FIG. 15A in accordance with an illustrative embodiment.

### DETAILED DESCRIPTION

Each and every feature described herein, and each and every combination of two or more of such features, is included within the scope of the present invention provided that the features included in such a combination are not mutually inconsistent.

While the present disclosure is directed to the practical assessment of biophysical signals, e.g., raw or pre-processed photoplethysmographic signals, biopotential/cardiac signals, etc., in the diagnosis, tracking, and treatment of cardiac-related pathologies and conditions, such assessment can be applied to the diagnosis, tracking, and treatment (including without limitation surgical, minimally invasive, lifestyle, nutritional, and/or pharmacologic treatment, etc.) of any pathologies or conditions in which a biophysical signal is involved in any relevant system of a living body. The assessment may be used in the controls of medical equipment or wearable devices or in monitoring applications (e.g., to report the wavelet-based features, parameters, or an intermediate output discussed herein).

The terms “subject” and “patient” as used herein are generally used interchangeably to refer to those who had undergone analysis performed by the exemplary systems and methods.

The term “cardiac signal” as used herein refers to one or more signals directly or indirectly associated with the structure, function, and/or activity of the cardiovascular system—including aspects of that signal's electrical/electrochemical conduction—that, e.g., cause contraction of the myocardium. A cardiac signal may include, in some embodiments, biopotential signals or electrocardiographic signals, e.g., those acquired via an electrocardiogram (ECG), the cardiac and photoplethysmographic waveform or signal capture or recording instrument later described herein, or other modalities.

The term “biophysical signal” as used herein includes but is not limited to one or more cardiac signal(s), neurological signal(s), ballistocardiographic signal(s), and/or photoplethysmographic signal(s), but it also encompasses more broadly any physiological signal from which information may be obtained. Not intending to be limited by example, one may classify biophysical signals into types or categories that can include, for example, electrical (e.g., certain cardiac and neurological system-related signals that can be observed, identified, and/or quantified by techniques such as the measurement of voltage/potential (e.g., biopotential), impedance, resistivity, conductivity, current, etc. in various domains such as time and/or frequency), magnetic, electromagnetic, optical (e.g., signals that can be observed, identified and/or quantified by techniques such as reflectance, interferometry, spectroscopy, absorbance, transmissivity, visual observation, photoplethysmography, and the like), acoustic, chemical, mechanical (e.g., signals related to fluid flow, pressure, motion, vibration, displacement, strain), thermal, and electrochemical (e.g., signals that can be correlated to the presence of certain analytes, such as glucose). Biophysical signals may in some cases be described in the context of a physiological system (e.g., respiratory, circulatory (cardiovascular, pulmonary), nervous, lymphatic, endocrine, digestive, excretory, muscular, skeletal, renal/urinary/excretory, immune, integumentary/exocrine and reproductive systems), one or more organ system(s) (e.g., signals that may be unique to the heart and lungs as they work together), or in the context of tissue (e.g., muscle, fat, nerves, connective tissue, bone), cells, organelles, molecules (e.g., water, proteins, fats, carbohydrates, gases, free radicals, inorganic ions, minerals, acids, and other compounds, elements, and their subatomic components. Unless stated otherwise, the term “biophysical signal acquisition” generally refers to any passive or active means of acquiring a biophysical signal from a physiological system, such as a mammalian or non-mammalian organism. Passive and active biophysical signal acquisition generally refers to the observation of natural or induced electrical, magnetic, optical, and/or acoustics emittance of the body tissue. Non-limiting examples of passive and active biophysical signal acquisition means include, e.g., voltage/potential, current, magnetic, optical, acoustic, and other non-active ways of observing the natural emittance of the body tissue, and in some instances, inducing such emittance. Non-limiting examples of passive and active biophysical signal acquisition means include, e.g., ultrasound, radio waves, microwaves, infrared and/or visible light (e.g., for use in pulse oximetry or photoplethysmography), visible light, ultraviolet light, and other ways of actively interrogating the body tissue that does not involve ionizing energy or radiation (e.g., X-ray). An active biophysical signal acquisition may involve excitation-emission spectroscopy (including, for example, excitation-emission fluorescence). The active biophysical signal acquisition may also involve transmitting ionizing energy or radiation (e.g., X-ray) (also referred to as “ionizing biophysical signal”) to the body tissue. Passive and active biophysical signal acquisition means can be performed in conjunction with invasive procedures (e.g., via surgery or invasive radiologic intervention protocols) or non-invasively (e.g., via imaging, ablation, heart contraction regulation (e.g., via pacemakers), catheterization, etc.).

The term “photoplethysmographic signal” as used herein refers to one or more signals or waveforms acquired from optical sensors that correspond to measured changes in light absorption by oxygenated and deoxygenated hemoglobin, such as light having wavelengths in the red and infrared spectra. Photoplethysmographic signal(s), in some embodiments, include a raw signal(s) acquired via a pulse oximeter or a photoplethysmogram (PPG). In some embodiments, photoplethysmographic signal(s) are acquired from off-the-shelf, custom, and/or dedicated equipment or circuitries that are configured to acquire such signal waveforms for the purpose of monitoring health and/or diagnosing disease or abnormal conditions. The photoplethysmographic signal(s) typically include a red photoplethysmographic signal (e.g., an electromagnetic signal in the visible light spectrum most dominantly having a wavelength of approximately 625 to 740 nanometers) and an infrared photoplethysmographic signal (e.g., an electromagnetic signal extending from the nominal red edge of the visible spectrum up to about 1 mm), though other spectra such as near-infrared, blue and green may be used in different combinations, depending on the type and/or mode of PPG being employed.

The term “ballistocardiographic signal,” as used herein, refers to a signal or group of signals that generally reflect the flow of blood through the entire body that may be observed through vibration, acoustic, movement, or orientation. In some embodiments, ballistocardiographic signals are acquired by wearable devices, such as vibration, acoustic, movement, or orientation-based seismocardiogram (SCG) sensors, which can measure the body's vibrations or orientation as recorded by sensors mounted close to the heart. Seismocardiogram sensors are generally used to acquire “seismocardiogram,” which is used interchangeably with the term “ballistocardiogram” herein. In other embodiments, ballistocardiographic signals may be acquired by external equipment, e.g., bed or surface-based equipment that measures phenomena such as a change in body weight as blood moves back and forth in the longitudinal direction between the head and feet. In such embodiments, the volume of blood in each location may change dynamically and be reflected in the weight measured at each location on the bed as well as the rate of change of that weight.

In addition, the methods and systems described in the various embodiments herein are not so limited and may be utilized in any context of another physiological system or systems, organs, tissue, cells, etc., of a living body. By way of example only, two biophysical signal types that may be useful in the cardiovascular context include cardiac/biopotential signals that may be acquired via conventional electrocardiogram (ECG/EKG) equipment, bipolar wide-band biopotential (cardiac) signals that may be acquired from other equipment such as those described herein, and signals that may be acquired by various plethysmographic techniques, such as, e.g., photoplethysmography. In another example, the two biophysical signal types can be further augmented by ballistocardiographic techniques.

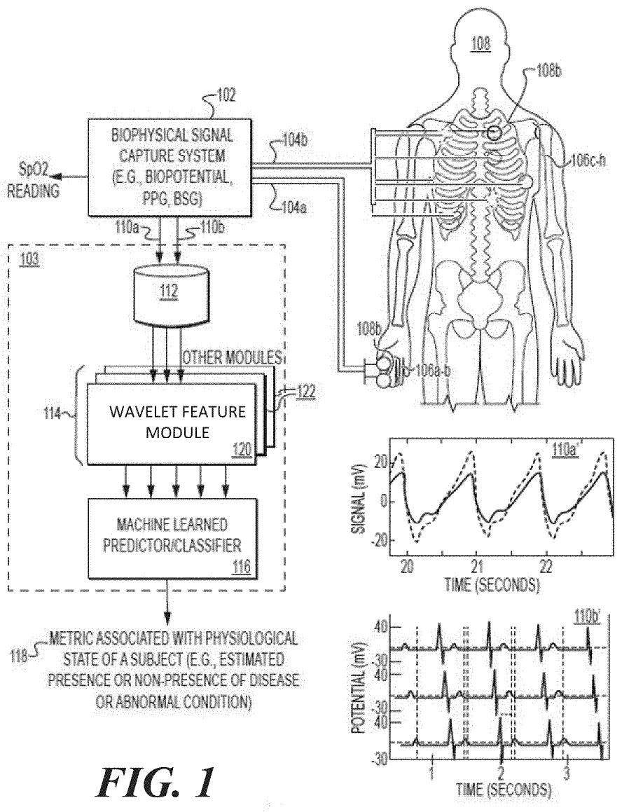

FIG. 1 is a schematic diagram of example modules, or components, configured to non-invasively compute wavelet-based features or parameters to generate, via a classifier (e.g., machine-learned classifier), one or more metrics associated with the physiological state of a patient in accordance with an illustrative embodiment. The modules or components may be used in a production application or the development of the wavelet-based features and other classes of features.

The example analysis and classifiers described herein may be used to assist a healthcare provider in the diagnosis and/or treatment of cardiac- and cardiopulmonary-related pathologies and medical conditions, or an indicator of one. Examples include significant coronary artery disease (CAD), one or more forms of heart failure such as, e.g., heart failure with preserved ejection fraction (HFpEF), congestive heart failure, various forms of arrhythmia, valve failure, various forms of pulmonary hypertension, among various other disease and conditions disclosed herein.

In addition, there exist possible indicators of a disease or condition, such as an elevated or abnormal left ventricular end-diastolic pressure (LVEDP) value as it relates to some forms of heart failure, abnormal left ventricular ejection fraction (LVEF) values as they relate to some forms of heart failure or an elevated mean pulmonary arterial pressure (mPAP) value as it relates to pulmonary hypertension and/or pulmonary arterial hypertension. Indicators of the likelihood that such indicators are abnormal/elevated or normal, such as those provided by the example analysis and classifiers described herein, can help a healthcare provider assess or diagnose that the patient has or does not have a given disease or condition. In addition to these metrics associated with a disease state of condition, other measurements and factors may be employed by a healthcare professional in making a diagnosis, such as the results of a physical examination and/or other tests, the patient's medical history, current medications, etc. The determination of the presence or non-presence of a disease state or medical condition can include the indication (or a metric of measure that is used in the diagnosis) for such disease.

In FIG. 1, the components include at least one non-invasive biophysical signal recorder or capture system 102 and an assessment system 103 that is located, for example, in a cloud or remote infrastructure or in a local system. Biophysical signal capture system 102 (also referred to as a biophysical signal recorder system), in this embodiment, is configured to, e.g., acquire, process, store and transmit synchronously acquired patient's electrical and hemodynamic signals as one or more types of biophysical signals 104. In the example of FIG. 1, the biophysical signal capture system 102 is configured to synchronously capture two types of biophysical signals shown as first biophysical signals 104a (e.g., synchronously acquired to other first biophysical signals) and second biophysical signals 104b (e.g., synchronously acquired to the other biophysical signals) acquired from measurement probes 106 (e.g., shown as probes 106a and 106b, e.g., comprising hemodynamic sensors for hemodynamic signals 104a, and probes 106c-106h comprising leads for electrical/cardiac signals 104b). The probes 106a-h are placed on, e.g., by being adhered to or placed next to, a surface tissue of a patient 108 (shown at patient locations 108a and 108b). The patient is preferably a human patient, but it can be any mammalian patient. The acquired raw biophysical signals (e.g., 106a and 106b) together form a biophysical-signal data set 110 (shown in FIG. 1 as a first biophysical-signal data set 110a and a second biophysical-signal data set 110b, respectively) that may be stored, e.g., as a single file, preferably, that is identifiable by a recording/signal captured number and/or by a patient's name and medical record number.

In the FIG. 1 embodiment, the first biophysical-signal data set 110a comprises a set of raw photoplethysmographic, or hemodynamic, signal(s) associated with measured changes in light absorption of oxygenated and/or deoxygenated hemoglobin from the patient at location 108a, and the second biophysical-signal data set 110b comprises a set of raw cardiac or biopotential signal(s) associated with electrical signals of the heart. Though in FIG. 1, raw photoplethysmographic or hemodynamic signal(s) are shown being acquired at a patient's finger, the signals may be alternatively acquired at the patient's toe, wrist, forehead, earlobe, neck, etc. Similarly, although the cardiac or biopotential signal(s) are shown to be acquired via three sets of orthogonal leads, other lead configurations may be used (e.g., 11 lead configuration, 12 lead configuration, etc.).

Plots 110a′ and 110b′ show examples of the first biophysical-signal data set 110a and the second biophysical-signal data set 110a, respectively. Specifically, Plot 110a′ shows an example of an acquired photoplethysmographic or hemodynamic signal. In Plot 110a′, the photoplethysmographic signal is a time series signal having a signal voltage potential as a function of time as acquired from two light sources (e.g., infrared and red-light source). Plot 110b′ shows an example cardiac signal comprising a 3-channel potential time series plot. In some embodiments, the biophysical signal capture system 102 preferably acquires biophysical signals via non-invasive means or component(s). In alternative embodiments, invasive or minimally-invasively means or component(s) may be used to supplement or as substitutes for the non-invasive means (e.g., implanted pressure sensors, chemical sensors, accelerometers, and the like). In still further alternative embodiments, non-invasive and non-contact probes or sensors capable of collecting biophysical signals may be used to supplement or as substitutes for the non-invasive and/or invasive/minimally invasive means, in any combination (e.g., passive thermometers, scanners, cameras, x-ray, magnetic, or other means of non-contact or contact energy data collection system as discussed herein). Subsequent to signal acquisitions and recording, the biophysical signal capture system 102 then provides, e.g., sending over a wireless or wired communication system and/or a network, the acquired biophysical-signal data set 110 (or a data set derived or processed therefrom, e.g., filtered or pre-processed data) to a data repository 112 (e.g., a cloud-based storage area network) of the assessment system 103. In some embodiments, the acquired biophysical-signal data set 110 is sent directly to the assessment system 103 for analysis or is uploaded to a data repository 112 through a secure clinician's portal.

Biophysical signal capture system 102 is configured with circuitries and computing hardware, software, firmware, middleware, etc., in some embodiments, to acquire, store, transmit, and optionally process both the captured biophysical signals to generate the biophysical-signal data set 110. An example biophysical signal capture system 102 and the acquired biophysical-signal set data 110 are described in U.S. Pat. No. 10,542,898, entitled “Method and Apparatus for Wide-Band Phase Gradient Signal Acquisition,” or U.S. Patent Publication No. 2018/0249960, entitled “Method and Apparatus for Wide-Band Phase Gradient Signal Acquisition,” each of which is hereby incorporated by reference herein in its entirety.

In some embodiments, biophysical signal capture system 102 includes two or more signal acquisition components, including a first signal acquisition component (not shown) to acquire the first biophysical signals (e.g., photoplethysmographic signals) and includes a second signal acquisition component (not shown) to acquire the second biophysical signals (e.g., cardiac signals). In some embodiments, the electrical signals are acquired at a multi-kilohertz rate for a few minutes, e.g., between 1 kHz and 10 kHz. In other embodiments, the electrical signals are acquired between 10 kHz and 100 kHz. The hemodynamic signals may be acquired, e.g., between 100 Hz and 1 kHz.

Biophysical signal capture system 102 may include one or more other signal acquisition components (e.g., sensors such as mechano-acoustic, ballistographic, ballistocardiographic, etc.) for acquiring signals. In other embodiments of the signal capture system 102, a signal acquisition component comprises conventional electrocardiogram (ECG/EKG) equipment (e.g., Holter device, 12 lead ECG, etc.).

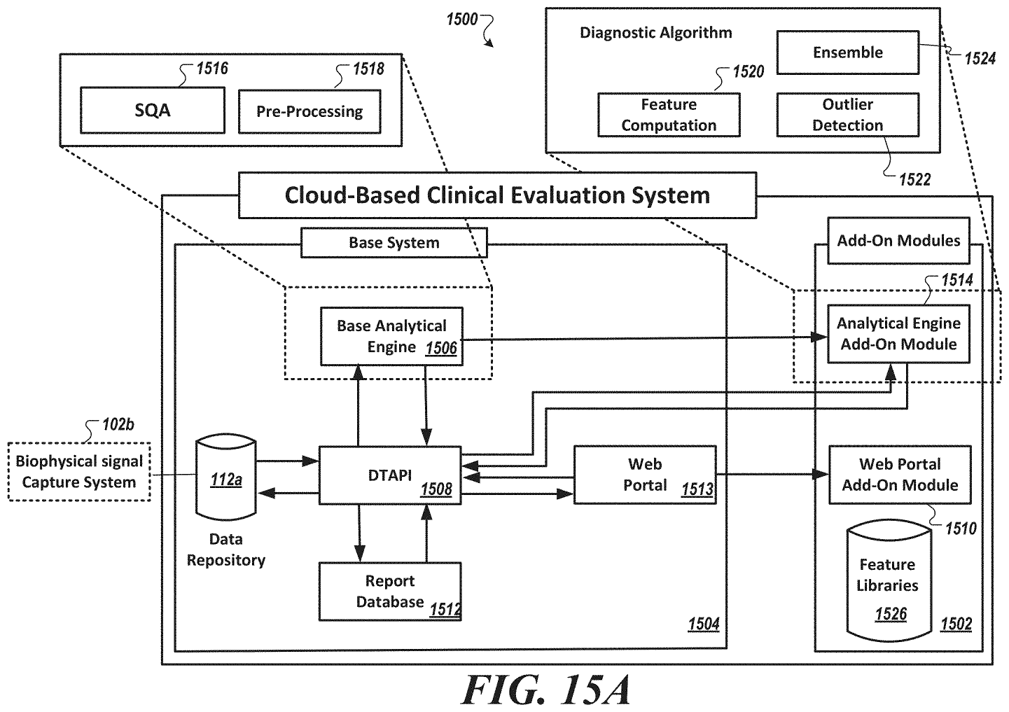

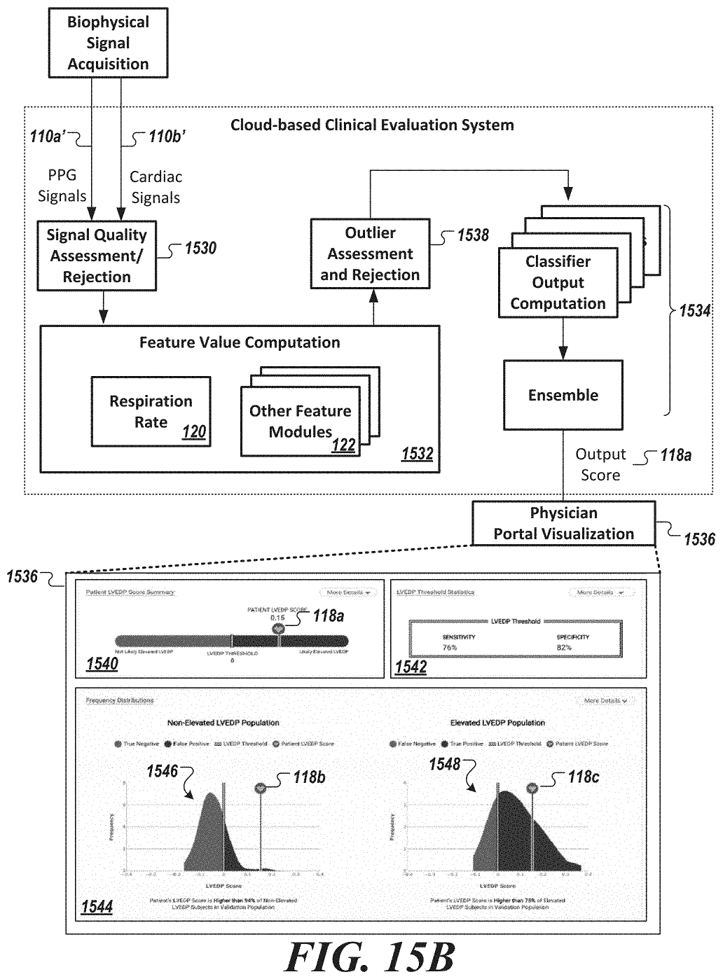

Assessment system 103 comprises, in some embodiments, the data repository 112 and an analytical engine or analyzer (not shown—see FIGS. 15A and 15B). Assessment system 103 may include feature modules 114 and a classifier module 116 (e.g., an ML classifier module). In FIG. 1, Assessment system 103 is configured to retrieve the acquired biophysical signal data set 110, e.g., from the data repository 112, and use it in the feature modules 114, which is shown in FIG. 1 to include a wavelet feature module 120 and other modules 122 (later described herein). The features modules 114 compute values of features or parameters, including those of wavelet-based features, to provide to the classifier module 116, which computes an output 118, e.g., an output score, of the metrics associated with the physiological state of a patient (e.g., an indication of the presence or non-presence of a disease state, medical condition, or an indication of either). Output 118 is subsequently presented, in some embodiments, at a healthcare physician portal (not shown—see FIGS. 15A and 15B) to be used by healthcare professionals for the diagnosis and treatment of pathology or a medical condition. In some embodiments, a portal may be configured (e.g., tailored) for access by, e.g., patients, caregivers, researchers, etc., with output 118 configured for the portal's intended audience. Other data and information may also be a part of output 118 (e.g., the acquired biophysical signals or other patient's information and medical history).

Classifier module 116 (e.g., ML classifier module) may include transfer functions, look-up tables, models, or operators developed based on algorithms such as but not limited to decision trees, random forests, neural networks, linear models, Gaussian processes, nearest neighbor, SVMs, Naïve Bayes, etc. In some embodiments, classifier module 116 may include models that are developed based on ML techniques described in U.S. Provisional Patent Application No. 63/235,960, filed Aug. 23, 2021, entitled “Method and System to Non-Invasively Assess Elevated Left Ventricular End-Diastolic Pressure”; U.S. Patent Publication No. 20190026430, entitled “Discovering Novel Features to Use in Machine Learning Techniques, such as Machine Learning Techniques for Diagnosing Medical Conditions”; or U.S. Patent Publication No. 20190026431, entitled “Discovering Genomes to Use in Machine Learning Techniques,” each of which is hereby incorporated by reference herein in its entirety.

Example Biophysical Signal Acquisition.

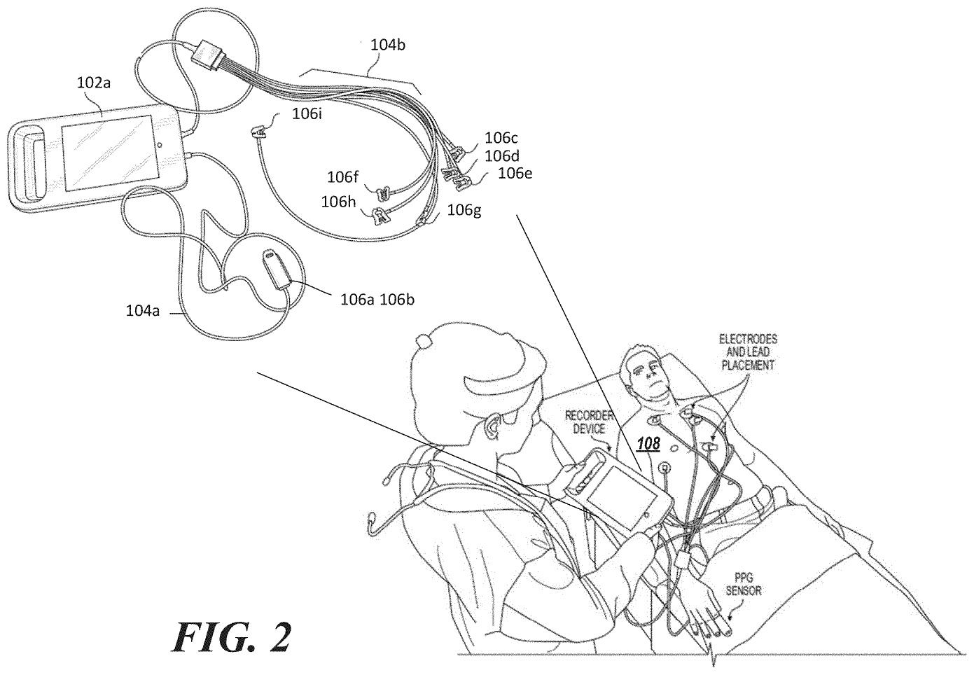

FIG. 2 shows a biophysical signal capture system 102 (shown as 102a) and its use in non-invasively collecting biophysical signals of a patient in a clinical setting in accordance with an illustrative embodiment. In FIG. 2, the biophysical signal capture system 102a is configured to capture two types of biophysical signals from the patient 108 while the patient is at rest. The biophysical signal capture system 102a synchronously acquires the patient's (i) electrical signals (e.g., cardiac signals corresponding to the second biophysical-signal data set 110b) from the torso using orthogonally placed sensors (106c-106h; 106i is a 7^{th} common-mode reference lead) and (ii) hemodynamic signals (e.g., PPG signals corresponding to the first biophysical-signal data set 110a) from the finger using a photoplethysmographic sensor (e.g., collecting signals 106a, 106b).

As shown in FIG. 2, the electrical and hemodynamic signals (e.g., 104a, 104b) are passively collected via commercially available sensors applied to the patient's skin. The signals may be acquired beneficially without patient exposure to ionizing radiation or radiological contrast agents and without patient exercise or the use of pharmacologic stressors. The biophysical signal capture system 102a can be used in any setting conducive for a healthcare professional, such as a technician or nurse, to acquire the requisite data and where a cellular signal or Wi-Fi connection can be established.

The electrical signals (e.g., corresponding to the second biophysical signal data set 110b) are collected using three orthogonally paired surface electrodes arranged across the patient's chest and back along with a reference lead. The electrical signals are acquired, in some embodiments, using a low-pass anti-aliasing filter (e.g., ˜2 kHz) at a multi-kilohertz rate (e.g., 8 thousand samples per second for each of the six channels) for a few minutes (e.g., 215 seconds). In alternative embodiments, the biophysical signals may be continuously/intermittently acquired for monitoring, and portions of the acquired signals are used for analysis. The hemodynamic signals (e.g., corresponding to the first biophysical signal data set 110a) are collected using a photoplethysmographic sensor placed on a finger. The photo-absorption of red light (e.g., any wavelengths between 600-750 nm) and infrared light (e.g., any wavelengths between 850-950 nm) are recorded, in some embodiments, at a rate of 500 samples per second over the same period. The biophysical signal capture system 102a may include a common mode drive that reduces common-mode environmental noise in the signal. The photoplethysmographic and cardiac signals were simultaneously acquired for each patient. Jitter (inter-modality jitter) in the data may be less than about 10 microseconds (μs). Jitter among the cardiac signal channels may be less than 10 microseconds, e.g., around ten femtoseconds (fs).

A signal data package containing the patient metadata and signal data may be compiled at the completion of the signal acquisition procedure. This data package may be encrypted before the biophysical signal capture system 102a transferring the package to the data repository 112. In some embodiments, the data package is transferred to the assessment system (e.g., 103). The transfer is initiated, in some embodiments, following the completion of the signal acquisition procedure without any user intervention. The data repository 112 is hosted, in some embodiments, on a cloud storage service that can provide secure, redundant, cloud-based storage for the patient's data packages, e.g., Amazon Simple Storage Service (i.e., “Amazon S3”). The biophysical signal capture system 102a also provides an interface for the practitioner to receive notification of an improper signal acquisition to alert the practitioner to immediately acquire additional data from the patient.

Example Method of Operation

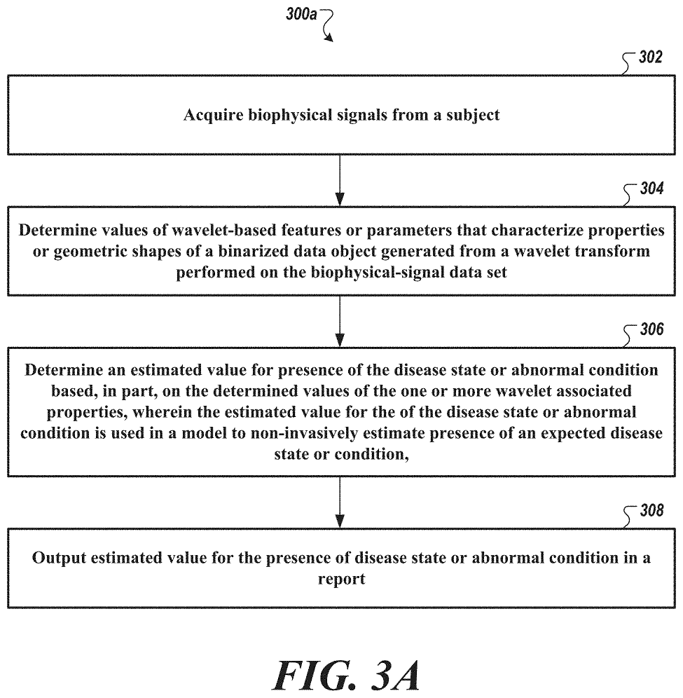

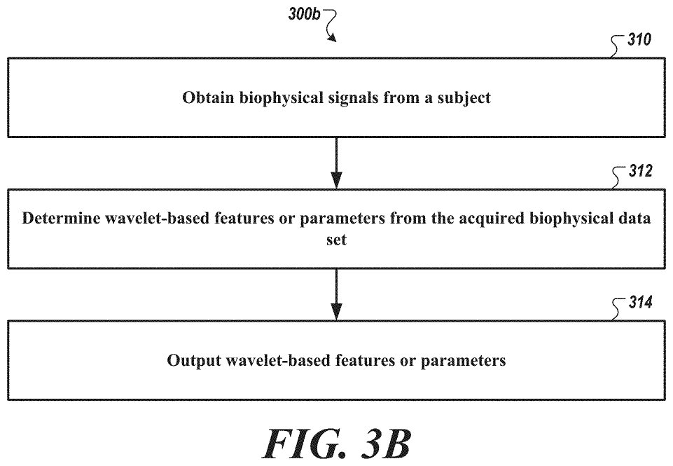

FIGS. 3A-3B each shows an example method to use wavelet-based features or their intermediate outputs in a practical application for diagnostics, treatment, monitoring, or tracking.

Estimation of Presence of Disease State or Indicating Condition. FIG. 3A shows a method 300a that employs wavelet-based parameters or features to determine estimators of the presence of a disease state, medical condition, or indication of either, e.g., to aid in the diagnosis, tracking, or treatment. Method 300a includes the step of acquiring (302) biophysical signals from a patient (e.g., cardiac signals, photoplethysmographic signals, ballistocardiographic signals), e.g., as described in relation to FIGS. 1 and 2 and other examples as described herein. In some embodiments, the acquired biophysical signals are transmitted for remote storage and analysis. In other embodiments, the acquired biophysical signals are stored and analyzed locally.

As stated above, one example in the cardiac context is the estimation of the presence of abnormal left-ventricular end-diastolic pressure (LVEDP) or mean pulmonary artery pressure (mPAP), significant coronary artery disease (CAD), abnormal left ventricular ejection fraction (LVEF), and one or more forms of pulmonary hypertension (PH), such as pulmonary arterial hypertension (PAH). Other pathologies or indicating conditions that may be estimated include, e.g., one or more forms of heart failure such as, e.g., heart failure with preserved ejection fraction (HFpEF), arrhythmia, congestive heart failure, valve failure, among various other disease and medical conditions disclosed herein.

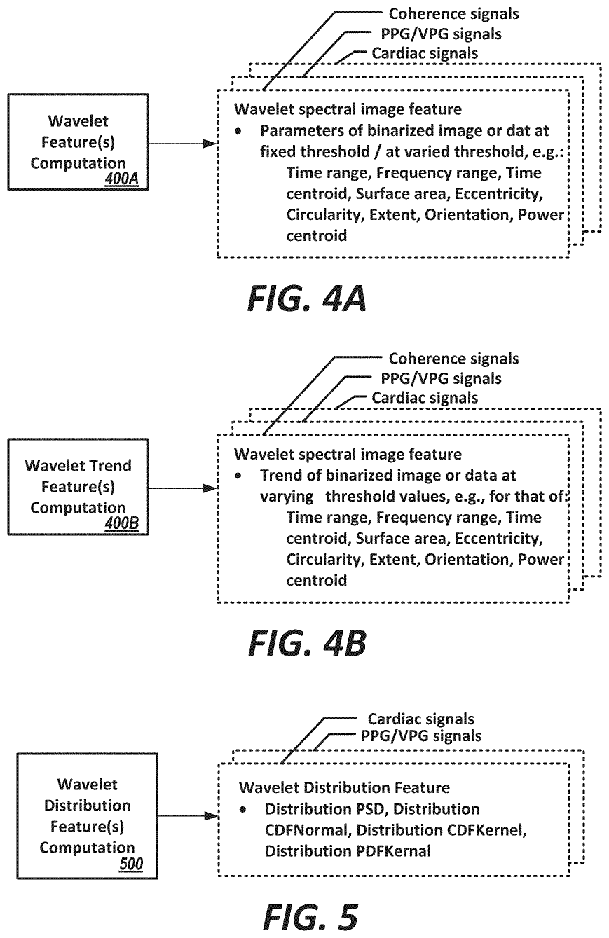

Method 300a further includes the step of retrieving (304) the data set and determining values of wavelet-based features or parameters that characterize properties or geometric shapes of a binarized data object generated from a wavelet transform performed on the biophysical-signal data set. Example operations to determine the values of wavelet-based features or parameters are provided in relation to FIGS. 4-14 later discussed herein. Method 300a further includes the step of determining (306) an estimated value for a presence of a disease state, medical condition, or an indication of either based on an application of the determined wavelet-based features to an estimation model (e.g., ML models). An example implementation is provided in relation to FIGS. 15A and 15B.

Method 300a further includes the step of outputting (308) estimated value(s) for the presence of disease state or abnormal condition in a report (e.g., to be used diagnosis or treatment of the disease state, medical condition, or indication of either), e.g., as described in relation to FIGS. 1, 15A, and 15B and other examples described herein.

Diagnostics or Condition Monitoring or Tracking using Wavelet-based Features or Parameters. FIG. 3B shows a method 300b that employs wavelet-based features or parameters or features for monitoring or controls of medical equipment or health monitoring device. Method 300b includes the step of obtaining (302) biophysical signals from a patient (e.g., cardiac signals, photoplethysmographic signals, ballistocardiographic signals, etc.). The operation may be performed continuously or intermittently, e.g., to provide output for a report or as controls for the medical equipment or the health monitoring device.

Method 300b further includes determining (310) wavelet-based features or parameters from the acquired biophysical data set, e.g., as described in relation to FIGS. 4-14. The determination based may be based on analysis of the continuously acquired signals over a moving window.

Method 300b further includes outputting (312) wavelet-based features or parameters (e.g., in a report for use in diagnostics or as signals for controls). As discussed herein, the wavelet-based features or parameters can provide a characterization or indication of the high-energy content of the spectral power in a given biophysical signal (e.g., biopotential/cardiac, photoplethysmographic signals, ballistocardiographic signals). For monitoring and tracking, the output may be via a wearable device, a handheld device, or medical diagnostic equipment (e.g., pulse oximeter system, wearable health monitoring systems) to provide augmented data associated with health. In some embodiments, the outputs may be used in resuscitation systems, cardiac or pulmonary stress test equipment, pacemakers, etc., in which frequency spectral information is desired.

Wavelet-Based Features or Parameters

FIGS. 4A, 4B, and 5 each shows an example wavelet-based feature computation module, for a total of three example modules configured to determine values of wavelet-based features or parameters in accordance with an illustrative embodiment. The wavelet feature computation module 400a and wavelet trend feature computation module 400b of FIGS. 4A and 4B, respectively, each determines features or parameters associated with spectral power of waveforms of interests (e.g., certain peaks or distinct regions) in a given cycle of an acquired biophysical signal (e.g., a photoplethysmographic and/or a biopotential/cardiac signal). The spectral power is determined, using a wavelet operator, as a spectral image or spectral data of frequency, time, and power for each cycle. Multiple spectral images from a corresponding number of cycles may be evaluated, and the statistical quantification of the results may be provided as the wavelet-based features or parameters. In Modules 400a, a single threshold is applied to the spectral image or spectral data for a given cycle to generate a binarized image or data (associated with high-power spectral content of the waveform of interest) to which time range, frequency range, time centroid, surface area, eccentricity, circularity, extent, orientation, and/or power centroid can be extracted from that binarized image or data as the wavelet-based feature or parameter. Module 400b evaluates the high-power spectral content of the waveforms by applying a set of thresholds to the spectral image or data to a trend, which is provided as the wavelet-based feature or parameter. The wavelet distribution computation module 500 of FIG. 5 assesses the power distribution (e.g., power spectral density, cumulative density function) of the spectral energy of the spectral image or spectral data of frequency, time, and power of various waveforms to which statistical quantification of the distributions are extracted as the wavelet-based features or parameters. The distribution of the power may be based on a power spectral density (PSD) distribution, a cumulative density function (CDF) distribution (e.g., CDFNormal or CDFKernel), or a power distribution function (PDF) distribution (e.g., PDFKernal).

A wavelet operator can generate wavelet series as a decomposition of the time series in an orthonormal space created by orthonormal wavelet basis functions constructed using a mother wavelet ψ. Mathematically, the continuous wavelet transform (CWT) of a real signal x(t), denoted by W here, can be calculated using Equation 1.

$$W ( a , τ ) = \frac{1}{\sqrt{a}} {\int }_{- ∞}^{+ ∞} x ( t ) ψ ( \frac{t - τ}{a} ) dt \qquad ( Equation 1 )$$

In Equation 1, a is the wavelet scale (similar to convolution kernel window size) and is the translation. While Fast Fourier Transform (FFT) is applicable for the decomposition of a stationary signal in the frequency domain, wavelet transform provides a flexible time-frequency analysis for non-stationary signals whose spectral content can change over time.

Discrete wavelet transform (DWT) is a class of wavelets that restricts the values of the scale and translation to computationally enhance the performance of the transformation with preserved accuracy. DWT commonly discretizes the scale into the power of 2 (a=1, 2, 4, 8, . . . ) and the translation to integer values as of the case in the discrete time-series (b=1, 2, 3, . . . ).

Examples of wavelet decomposition that may be used include, but are not limited to, continuous wavelet transforms (CWT), and discrete wavelet transforms (DWT).

### Example #1—Wavelet-Based Features

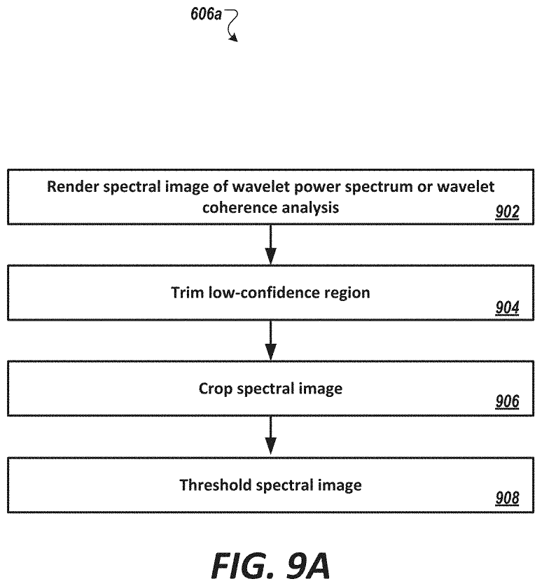

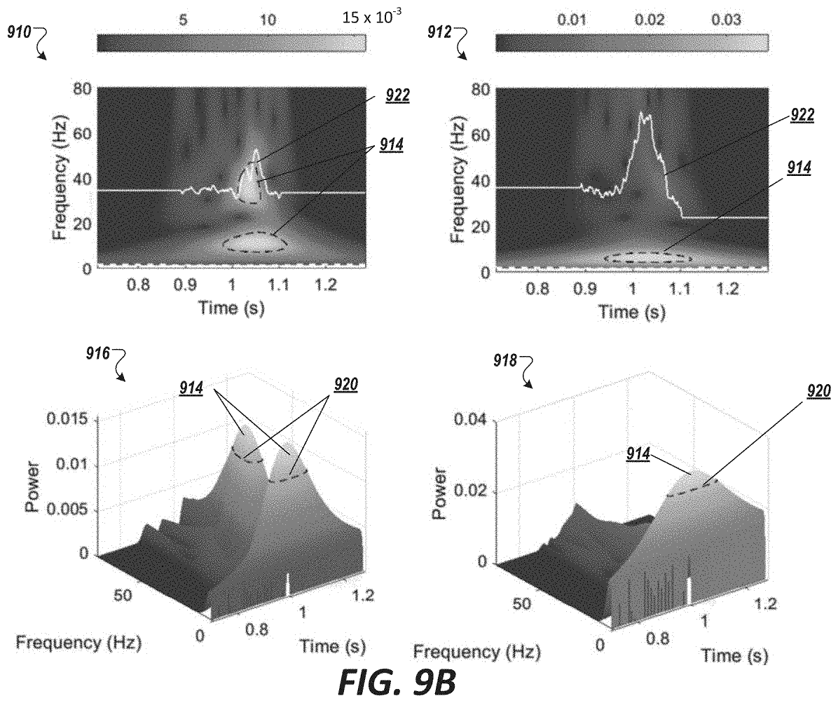

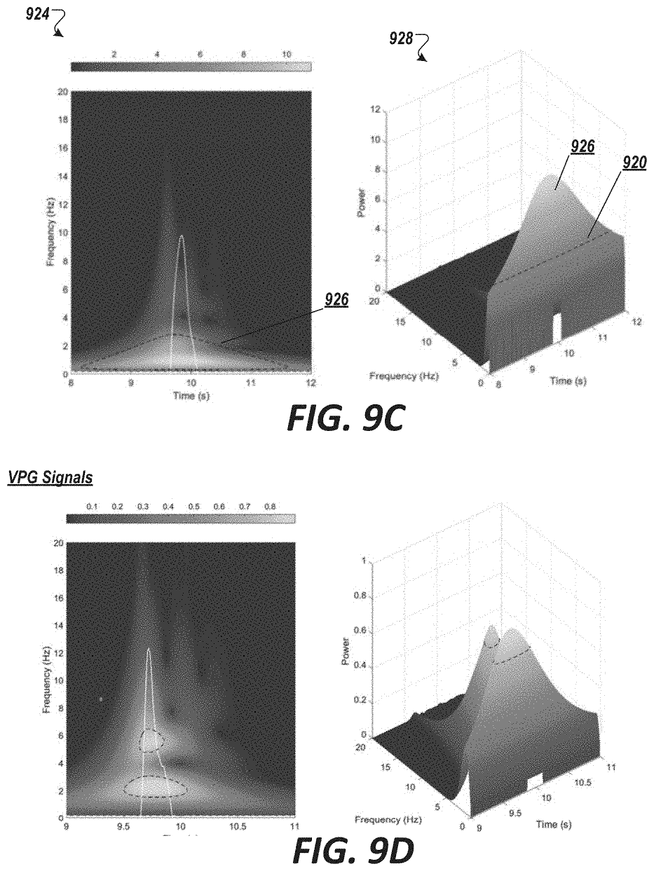

FIG. 4A illustrates as the first of three example feature or parameter categories, an example wavelet feature computation module 400a configured to extract high spectral energy characteristics of a waveform region of interest in a given biophysical signal. A spectral image or spectral data is generated from a wavelet transformation of a given waveform region of interest to which a threshold operator is applied to generate a binarized spectral image or data corresponding the high spectral power characteristics of the waveform region. The computation is preferably performed over multiple heart cycles to extract, as wavelet-based features or parameters, a statistical characterization of the time range, frequency range, time centroid, surface area, eccentricity, circularity, extent, orientation, and/or power centroid of the high spectral energy characteristics of a waveform region. FIGS. 9A-9D show example methods of the wavelet feature module of FIG. 4A to generate wavelet-based features from a binarized spectral image of the spectral wavelet model of a cardiac signal, a photoplethysmographic signal, and a velocityplethysmogram signal, in accordance with an illustrative embodiment.

In implementations in which Module 400a generates spectral images, the images may be represented in two-dimensional axes of time and frequency, and the power is shown as color. To this end, a threshold operator may be used to produce a binarized image with all power above a specific level are assigned the same color and the remaining power assigned a different color. The analysis of spectral energy as images allows image processing operations to be used that can readily extract visually discernable characteristics from the spectral image.

Table 1 shows an example set of 11 extractable high spectral energy characteristics of a waveform region of interest from a generated binarized spectral image or data.

TABLE 1

| Feature Name | Feature Description |
|---|---|
| Time range | The time duration of a high spectral energy region of the waveform of |
| | interest in a biophysical signal, the time duration, e.g., corresponding to |
| | a length (e.g., of the x-dimension) of a bounding box generated around |
| | a thresholded object corresponding to the high spectral energy region in |
| | a binarized spectral image or data. |
| Frequency range | The frequency range of a high spectral energy region of the waveform |
| | of interest in a biophysical signal, the frequency range, e.g., |
| | corresponding to the height (e.g., of the y-dimension) of a bounding box |
| | generated around a thresholded object corresponding to the high |
| | spectral energy region in the binarized spectral image or data. |
| Time centroid | The center of mass of the high spectral energy region of the waveform |
| | of interest in a biophysical signal, the center of mass determined from |
| | a binarized thresholded region in the time dimension (e.g., x-axis). |
| Frequency centroid | The center of mass of the high spectral energy region of the waveform |
| | of interest in a biophysical signal, the center of mass determined from |
| | a binarized thresholded region in the frequency dimension (e.g., y-axis). |
| Surface area | The size of the high spectral energy region of the waveform of interest |
| | in a biophysical signal, the size determined from a binarized |
| | thresholded region (e.g., in pixel) of the binarized spectral image. |
| Eccentricity | The eccentricity of the shape of the high spectral energy region of the |
| | waveform of interest in a biophysical signal, the eccentricity is |
| | determined as a ratio of the distance between (i) the foci of a fitted |
| | ellipse enclosing the binarized region and (ii) its major axis length (e.g., |
| | having a value between 0 and 1). An ellipse having an eccentricity of |
| | 0 is a circle, while an ellipse whose eccentricity is 1 is a line segment. |
| Circularity | The circularity of the shape of the high spectral energy region of the |
| | waveform of interest in a biophysical signal, the circularity determined as |
| | $\frac{4 \times surface Area \times π}{{Perimeter}^{2}} .$ For a circle, the circularity has a value of l. |
| Extent | The extent of the shape of the high spectral energy region of the |
| | waveform of interest in a biophysical signal, the extent determined as a |
| | ratio of pixels in the binarized thresholded region to a number of pixels |
| | in a bounding box. |
| Orientation | The orientation of the shape of the high spectral energy region of the |
| | waveform of interest in a biophysical signal, the orientation being |
| | determined as an angle between the x-axis and the major axis of a fitted |
| | ellipse encompassing the binarized region (e.g., ranged from −90° to |
| | 90°). |
| Power centroid | The center of mass of the high spectral energy region of the waveform |
| | of interest in a biophysical signal, the power centroid being determined |
| | from a binarized thresholded region in the power dimension (e.g., z- |
| | axis or color). |
| NumRegions | The number of high spectral energy regions of the waveform of interest |
| | in a biophysical signal as determined from a number of binarized |
| | thresholded regions. |

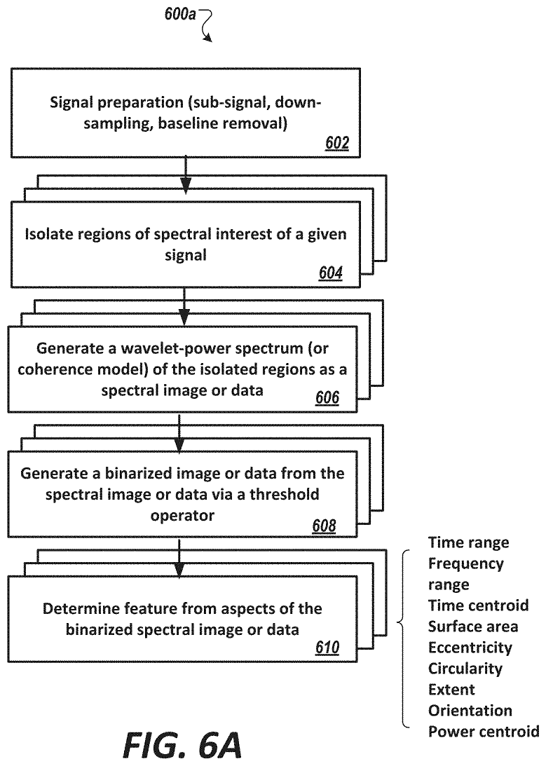

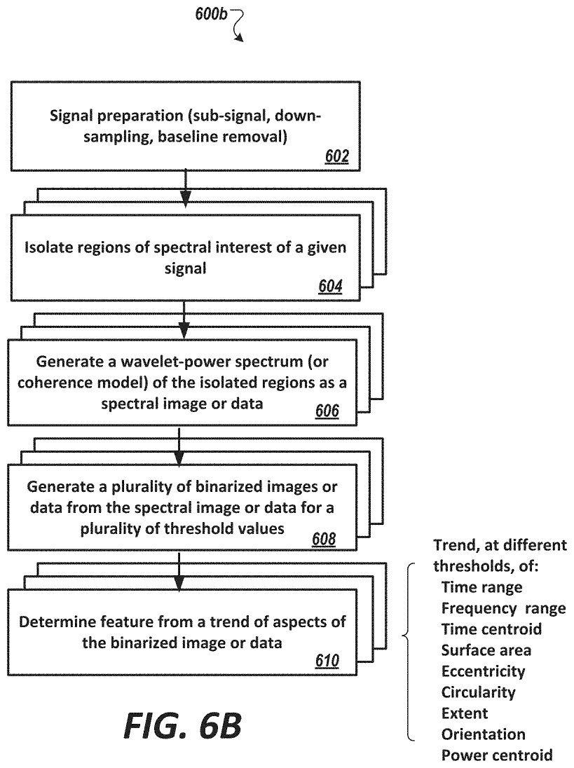

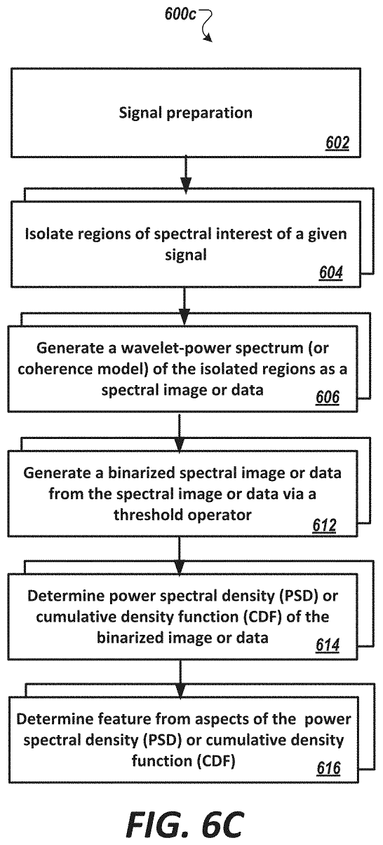

FIG. 6A shows a method 600a to generate wavelet-based features or parameters, e.g., as performed by the wavelet feature computation module 400a of FIG. 4A, in accordance with an illustrative embodiment, which can be used wholly, or partially, to generate wavelet-based features or parameters and its outputs to be used in machine-learned classifier to determine a metric associated with a physiological system of a subject under study. To determine the features of Table 1, Module 400a is configured, in some embodiments, to (i) pre-process (602) the acquired biophysical signal, (ii) isolate (604) waveform regions of interest of a given signal, (iii) generate (606) a wavelet-power spectrum (or coherence model) of the isolated regions as a spectral image or data, (iv) generate (608) a binarized spectral image or data from the spectral image or data via a threshold operator, and (v) determine (610) a feature from aspects of the binarized spectral image or data.

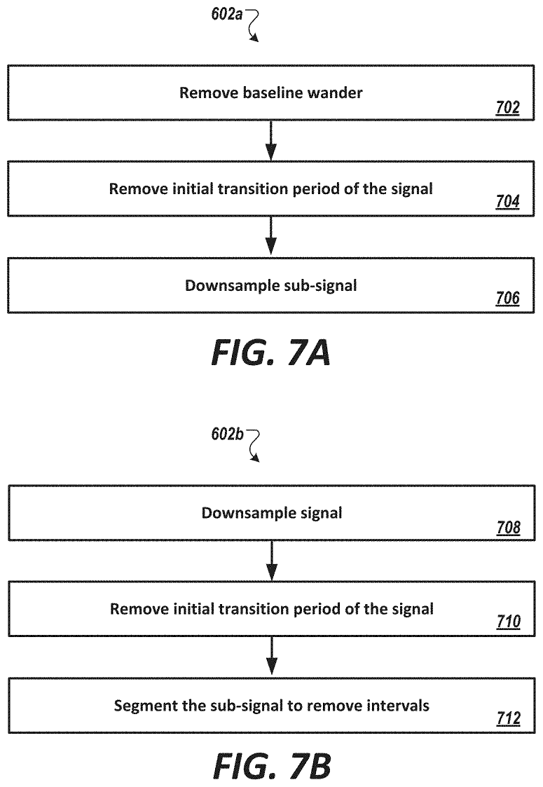

In FIG. 6A, the method 600a includes pre-processing (602) the acquired biophysical signals. The pre-processing 602 operation may include sub-signaling, down-sampling, and/or baseline removal, e.g., to improve the accuracy of the analysis or improve the computation efficiency. Sub-signaling includes the removal of the first or other portions of the signal to eliminate initial or other transition periods. FIG. 7A shows an example method 602 (shown as 602a) of pre-processing a cardiac signal in accordance with an illustrative embodiment. In FIG. 7A, the pre-processing operation 602a (i) removes (702) the baseline wandering, (ii) removes (704) the first x seconds of the signal (e.g., 30 seconds) to eliminate the initial transition period, and then (iii) down-samples (706) the signal (e.g., from 8 kHz to 500 Hz). FIG. 7B shows an example method 602 (shown as 602b) of pre-processing a photoplethysmographic signal in accordance with an illustrative embodiment. In FIG. 7B, the pre-processing operation 602b (i) down-samples (708) an acquired signal to 125 Hz, (ii) removes (710) the first x second the signal (e.g., 30 seconds), (iii) segments (712) the signal into windows (e.g., 20-seconds segments with 10-seconds skip intervals).

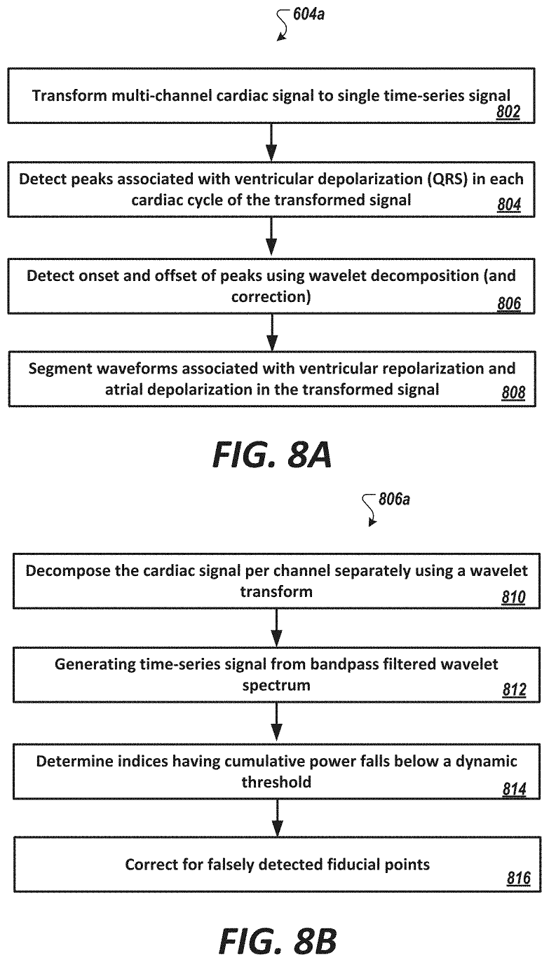

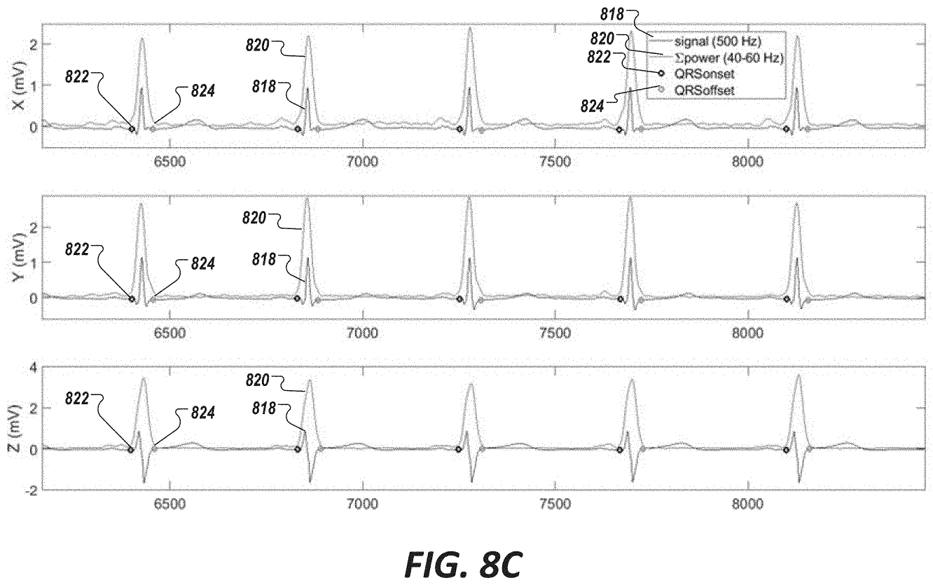

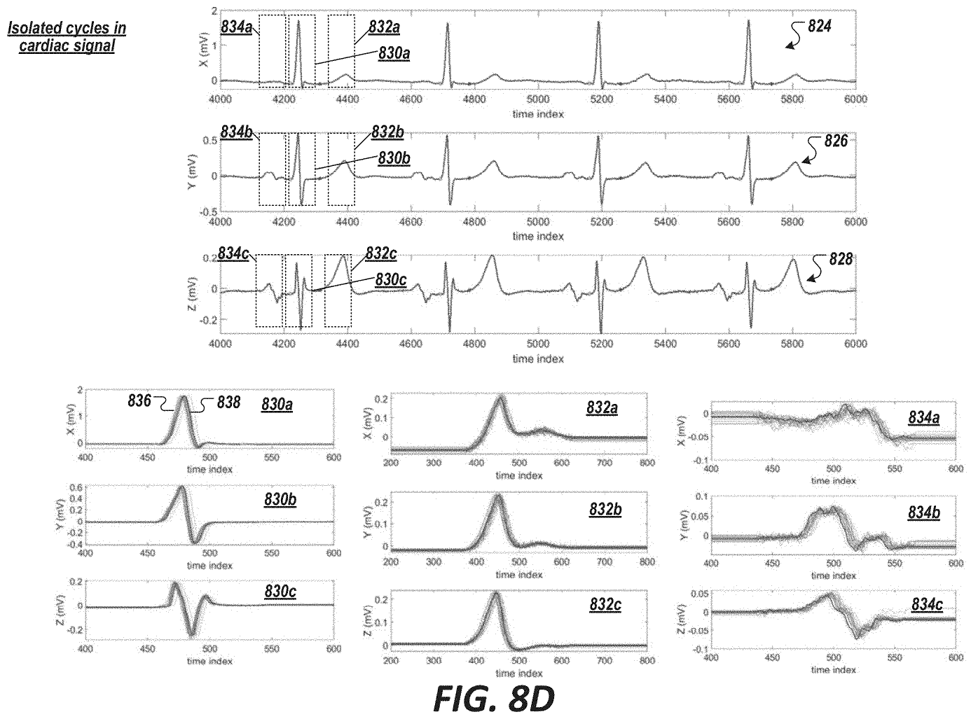

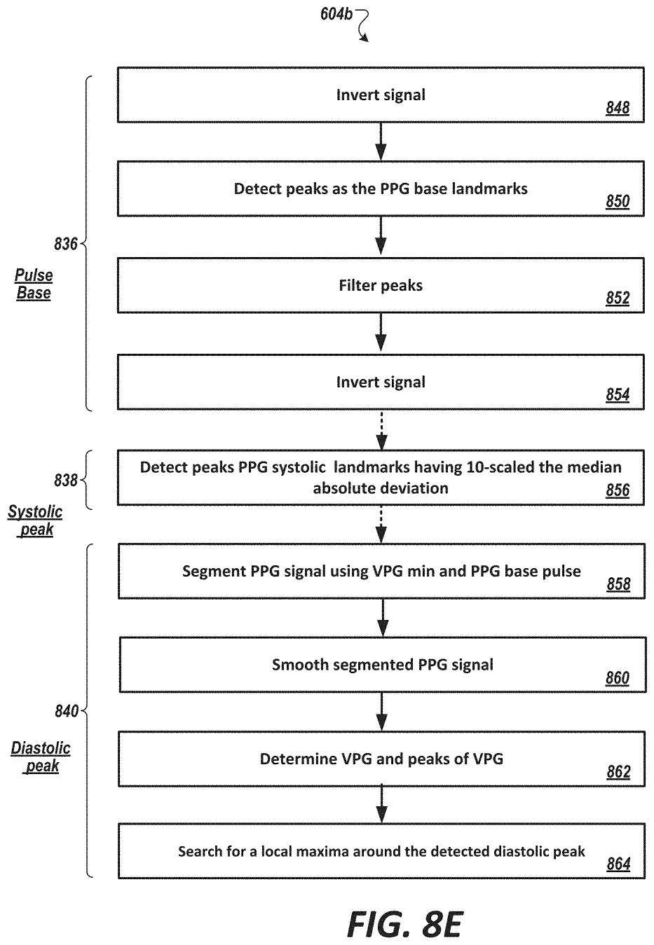

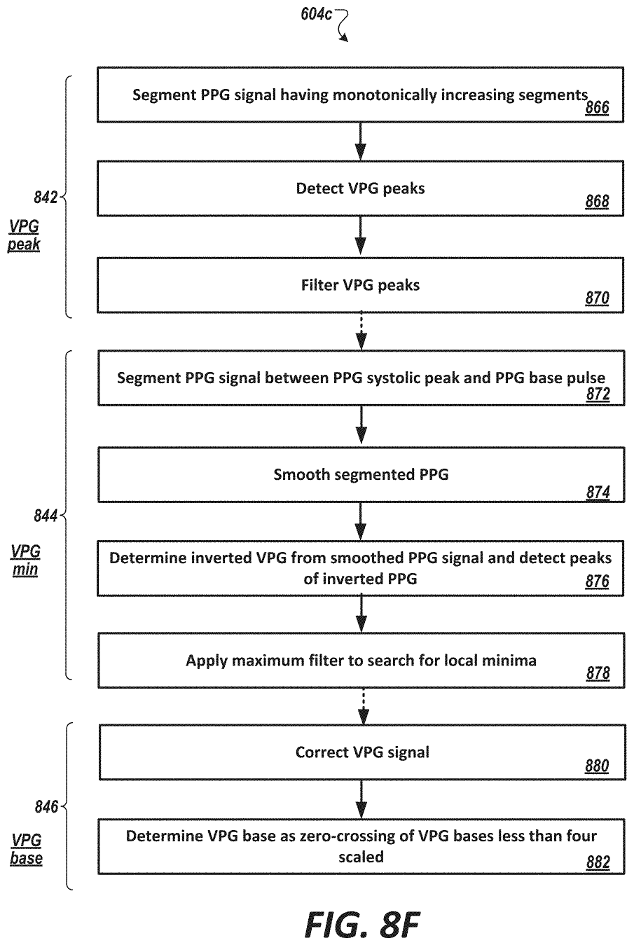

The method 600a then includes isolating or detecting (604) (also referred to as delineating), e.g., using wavelet transform, one or more regions of spectral interest in the biophysical signals. The waveform region detection 604 facilitates the isolation of specific waveform regions for analysis that can minimize the influences of other beats and temporal noises on the power spectrum analysis. Wavelet operators facilitate a more accurate analysis of the waveform frequency components as they can account for frequency drift over time. For cardiac signals, the detection operation (604) may include isolating one or more regions of the cardiac waveform, e.g., associated with atrial depolarization (also referred to as “P wave”), ventricular depolarization (also referred to as QRS wave or peaks), and/or ventricular repolarization (also referred to as “T wave”). For photoplethysmographic signals, the detection operation (604) may include isolating waveforms regions associated with the base, peak, and minimum landmarks in the photoplethysmographic signal(s) or velocityplethysmogram signal(s) derived from the photoplethysmographic signal(s). Further description of the delineation operation 604 is provided in the description provided in relation to FIGS. 8A-8F.

Spectral Image or Data Generation. The method 600a then includes generating (606), using wavelet decomposition/transform operation, a wavelet-power spectrum model of the isolated regions as a spectral image or spectral data. The power spectrum may be calculated for each or a substantial portion (e.g., >50%) of the isolated waveforms (i.e., P-wave. T-wave, and QRS complex of a cardiac signal, a coherence waveform, or a peak or landmark within a photoplethysmographic or velocityplethysmogram signal) for the plurality of cycles in a given biophysical signal. For continuous real-time implementations (e.g., for FIG. 3B), the transform may be performed on a current window of the acquired signals.

The cardiac, photoplethysmographic, and/or coherence power spectrum may be calculated for each of the detected waveforms (e.g., P-wave. T-wave, QRS complex of a cardiac signal or peak or landmark within a photoplethysmographic or velocityplethysmogram signal, etc.). In some embodiments, a 1-D continuous wavelet transform is applied, such as a Morlet (also referred to as a Gabor) wavelet as the mother wavelet. Other wavelets, e.g., having equal variance in time and frequency, may be used, e.g., Gaussian, Mexican Hat, Spline, and Mayer wavelet. The wavelet may have a resolution of 48 voices per octave. The Morlet wavelet is a wavelet composed of a complex exponential (carrier) multiplied by a Gaussian window (envelope), as shown in Equation 2.

ψ(t)=exp(iωt)exp(−t²/2σ²) (Equation 2)

In Equation 2, ω is the wavelet central frequency, and σ=n/2πf is the width of the Gaussian window with n (the number of cycles) controlling the time-frequency resolution trade-off.

Coherence waveforms may be determined as a measure of the correlation between time series signals, such as between two channels in a cardiac signal data set or between two channels in a photoplethysmographic signal data set or between two channels of different modalities. Wavelet coherence may be determined, for example, by Equation 3.

$${C}_{w} = \frac{{C}_{xy} ( a , τ )}{{\vert S ( {C}_{x} ( a , τ ) ) \vert}^{2} \cdot {\vert S ( {C}_{y} ( a , τ ) ) \vert}^{2}} \qquad ( Equation 3 )$$

In Equation 3, the cross-spectrum C_{xy} is a measure of the distribution of power of two signals x and y in the time-frequency domain given by Equation 4.

C_{xy}(a,τ)=|S(C*_{xy}(a,τ))|² (Equation 4)

In Equation 4, superscript denotes a complex conjugate, and S is a smoothing operator in time and scale. In some embodiments, a coherence spectrum operator is performed, e.g., with 32 voices per octave to find the coherence spectrum of the paired channels (e.g., between channels X and Y, channels x and X, and channels Y and Z). Coherence spectrum may be generated between a cardiac signal and a photoplethysmographic signal.

High-Power Spectral Image or Data Generation. Referring to FIG. 6A, the method 600a includes generating (608) a binarized spectral image of the spectral image or spectral data of the high-power spectral content of a waveform signal of interest. Method 606 may generate the high-power spectral images or spectral data for an atrial depolarization waveform (e.g., P-wave spectral image), a ventricular depolarization waveform (e.g., QRS spectral image), a ventricular repolarization waveform (e.g., T-wave spectral image), a systolic waveform of a photoplethysmographic signal, a diastolic waveform of a photoplethysmographic signal, a pulse base waveform of a photoplethysmographic signal, a peak waveform of a velocityplethysmogram signal, a minimum waveform of a velocityplethysmogram signal, a base waveform of a velocityplethysmogram signal. High-power spectral images or spectral data may also be generated from a coherence analysis.

FIG. 9A is an example implementation of the method 606 (shown as 606a) to generate a binarized spectral image or data of the spectral wavelet model in accordance with an illustrative embodiment. The method 606a includes rendering (902) a spectral image (e.g., three-dimensional data) of frequency, time, and power from a power spectral or coherence analysis.

The method 606a includes, in some embodiments, trimming (904) the generated spectral image or data to remove regions in the image or data where confidence in the wavelet transformation is low, e.g., due to wavelet cone of influence. These regions are typically at very low frequencies and at the temporal boundaries.

The operation 606a then includes (i) cropping (906) the spectral image temporally to limit to the central time period of the image and (ii) cropping (906) the image in the frequency domain to limit to a maximum frequency. For cardiac signals, the image may be cropped temporarily (e.g., in the temporal/time axis, e.g., x-axis) to limit the image to the central 0.6 seconds. The image may be cropped in the frequency domain to limit to a maximum frequency. The maximum frequency may be applied differently for the different waveform regions. For example, cropped maximum frequency for the P-wave waveforms may be 60 Hz; for the QRS waveforms, 80 Hz; and for the T-wave waveforms, 60 Hz. The QRS waveforms may be permitted higher frequencies to facilitate analysis of the higher frequency content as compared to P-waves and T-waves.

The operation 606a includes thresholding (908) the image to generate a binarized logical representation of the spectral characteristics of the acquired signal. In some embodiments, the image is thresholded using percentiles where all values (corresponding to spectral power in the image) above a certain percentile is maintained, and all power below that threshold is removed. Table 2 shows example threshold levels for various cardiac waveform types.

TABLE 2

| Waveform type | Threshold levels |
|---|---|
| Atrial depolarization (P-wave) image | The threshold for top 1% |
| Ventricular depolarization (QRS-wave) | The threshold for top 1% |
| image | |
| Ventricular repolarization (T-wave) image | The threshold for top 2.5% |

As shown in Table 2, thresholding may be dependent on the waveform, e.g., with the top 1% (i.e., a threshold of 0.01) used for the P-wave and QRS-wave, and 2.5% (i.e., a threshold of 0.025) for the T-wave. The lower threshold facilitates analysis of the power in more of the lower frequency components associated with the ventricular repolarization (T-wave). The thresholded regions in the binarized spectral image or data thus include the high-power regions of the spectral image in reference to its frequency location and time location within the acquired biophysical signal.

FIG. 9B shows two example wavelet spectral images (shown as 910, 912) and associated binarized elements (shown as 914) generated from a spectral wavelet model according to the method of FIG. 9A in accordance with an illustrative embodiment. In FIG. 9B, the wavelet spectral images (910, 912) are each shown as a time-frequency plot in which time is provided in the x-axis and frequency is provided in the y-axis.

In FIG. 9B, the wavelet spectral image 910 is shown to have multiple peaks, and the wavelet spectral image 912 is shown to have a single distinguishable peak. FIG. 9B also shows the corresponding three-dimensional data space (916 and 918) used to generate the images 910, 912. In FIG. 9B, the three-dimensional data space is shown as a time-frequency-power plot in which time is provided in the x-axis, frequency is provided in the y-axis, and power is provided in the z-axis. A convex hull encapsulation (shown as 920) is also shown that bounds each of the binarized regions above a pre-defined threshold to which features, and their associated values, can be determined. As noted above, though the analysis is presented with respect to images, they may be performed on a similar data space that is not in a visual format.

FIG. 9B shows the spectral wavelet model of a channel of a cardiac signal. In FIG. 9B, the cardiac signal waveform region 922 used to generate the spectral image is shown superimposed over the respective wavelet spectral image. The signal 922 is shown in time versus amplitude. FIG. 9C shows an example wavelet spectral image (shown as 924) and associated binarized elements (926) generated from a spectral wavelet model of a photoplethysmographic signal according to the method of FIG. 9A. FIG. 9C also shows the corresponding three-dimensional data space (928) used to generate the image 924. FIG. 9D shows an example wavelet spectral image and associated binarized elements generated from a spectral wavelet model of a velocityplethysmogram signal data set according to the method of FIG. 9A.

Features Computation. Referring to FIG. 6A, the method 600a includes determining (610) features, and associated values from aspects of the generated binarized spectral image or data by detecting or quantifying properties or parameters in the binarized spectral image or data. To quantify the properties or parameters in the spectral image or data, Module 400a (and/or Module 400b) may assign a bounding box to each distinct region in the spectral image or data using a convex hull operator or any operator that can encapsulate regions. Where multiple distinct regions are identified, the computed feature values may be determined as a weighted average of the values across the multiple regions. For image data, the weighted average may be based on the surface area/pixel counts of the image. For data, the weighted average may be based on data points. Module 400a (and/or module 400b) may exclude/denoise regions with very small surface areas from participating in the feature calculation.

Cardiac signal Spectral-Image based features. Table 3A shows a summarized set of 198 features directed to 11 geometric or properties (shown as “TimeRange,” “TimeCentroid,” FrequencyCentroid,” “SurfaceArea,” “Eccentricity,” “Circularity,” and “Extent” in Table 3A) that can be extracted from high-power spectral content information in a binarized spectral image or data for a respective waveform signal region of interests (e.g., atrial depolarization (P-wave) regions, ventricular depolarization (QRS) wave regions, and ventricular repolarization (T-wave) regions in a given signal channel of a cardiac signal (referred signal channels X, Y, and/or Z in Table 3A) to provide up to 99 features. The wavelet-based feature or parameter may be computed from spectral images generated across many cardiac cycles. To compress the computed feature values to a summary feature (e.g., suitable for ML), Module 400a may employ (i) the interquartile range (“IQR”) that shows how the feature varies across the cycles or (ii) the middle value in the distribution of feature values (“median”). When quantified for the IQR or the median, Module 400a can compute, e.g., for a total of 198 potential wavelet-based features or parameters.

It has been observed through experimentation that time range, frequency range, time centroid, frequency centroid, surface area, eccentricity, circularity, extent, orientation, and power centroid among the various waveform signal regions and signal channels have significant utility in the assessment of the presence or non-presence of at least one cardiac disease or condition—specifically, the determination of presence or non-presence of elevated LVEDP. It has also been observed through experimentation that time range, frequency range, time centroid, frequency centroid, surface area, eccentricity, circularity, extent, and orientation among the various waveform signal regions and signal channels have significant utility in the assessment of the presence or non-presence of coronary artery disease. The list of the specific wavelet-based features or parameters determined to have significant utility in the assessment of the presence or non-presence of abnormal or elevated LVEDP and the presence or non-presence of significant CAD is provided in Table 8A and 9A, respectively.

TABLE 3A

| Signal Region | | Signal Channel | |
|---|---|---|---|
| to which | | to which | |
| Features | | features | Distribution |
| are extracted | Feature Name | are extracted | Summary |
| wtPwave | TimeRange*^{,} ** | X | Iqr |
| wtQRSwave | FrequencyRange** | Y | Median |
| wtTwave | TimeCentroid*^{,} ** | Z | |
| | FrequencyCentroid*^{,} ** | | |
| | SurfaceArea*, ** | | |
| | Eccentricity*^{,} ** | | |
| | Circularity*^{,} ** | | |
| | Extent*^{,} ** | | |
| | Orientation*^{,} ** | | |
| | PowerCentroid* | | |
| | Numregions | | |

PPG signal Spectral-Image-based features. Table 3B shows an example set of 264 extractable wavelet-based features or parameters of the same 11 parameters for three example regions of two photoplethysmographic signals and three example regions of a corresponding two set of velocityplethysmogram signals, to provide up to 132 features. The parameters may be extracted across multiple photoplethysmographic signal cycles or velocityplethysmogram signal cycles and can be quantified as the interquartile range (“IQR”) or the median to provide a total of 264 features. It also has been observed through experimentation that time range, frequency range, time centroid, frequency centroid, surface area, eccentricity, circularity, extent, orientation, power centroid, and number of regions, among PPG and VPG channels have significant utility in the assessment of the presence or non-presence of coronary artery disease. The list of the specific wavelet-based features or parameters determined to have significant utility in the assessment of presence or non-presence of significant CAD is provided in Table 9A.

TABLE 3B

| Signal region | | Signal channel | |
|---|---|---|---|
| to which | | to which | |
| features | | features | Distribution |
| are extracted | Feature Name | are extracted | Summary |
| Systolic peak (for | timeRange** | PPG_IR | Iqr |
| PPG) | | | |
| Diastolic peak | frequencyRange** | PPG_red | median |
| (for PPG) | | | |
| Pulse bases (for | timeCentroid** | VPG_IR | |
| PPG) | | | |
| Maximum peak | FrequencyCentroid** | VPG_red | |
| (for VPG) | | | |
| Minimum peak | surfaceArea | | |
| (for VPG) | | | |
| Base (for VPG) | Eccentricity** | | |
| | Circularity** | | |
| | Extent** | | |
| | Orientation** | | |
| | powerCentroid** | | |
| | Numregions** | | |

Coherence analysis Spectral-Image features. Table 3C shows an example set of 33 extractable wavelet-based features or parameters for the 11 parameters of Table 1 as computed for three coherence waveforms, namely, among cardiac signals, XY, XZ, and YZ. It has been observed through experimentation that the frequency centroid parameter and the eccentricity parameter have significant utility in the assessment of the presence or non-presence of at least one cardiac disease or condition—specifically, the determination of presence or non-presence of elevated LVEDP. It also has been observed through experimentation that frequency range, time centroid, frequency centroid, circularity, and extent parameters have significant utility in the assessment of the presence or non-presence of coronary artery disease. The list of the specific wavelet-based features or parameters determined to have significant utility in the assessment of the presence or non-presence of abnormal or elevated LVEDP and the presence or non-presence of significant CAD is provided in Tables 8A and 9A, respectively.

TABLE 3C

| Signal waveform to which | |
|---|---|
| features are extracted | Features |
| Coherence XY | timeRange |
| Coherence XZ | frequencyRange** |
| Coherence YZ | timeCentroid |
| | FrequencyCentroid*^{,} ** |
| | surfaceArea |
| | Eccentricity* |
| | circularity** |
| | Extent** |
| | Orientation |
| | powerCentroid |
| | Numregions |

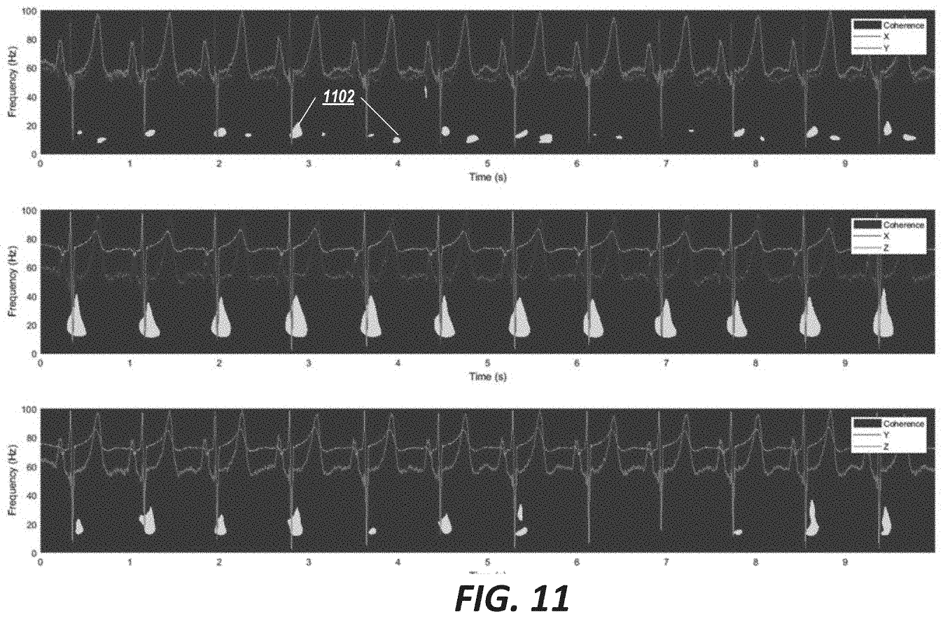

FIG. 11 shows example binarized wavelet spectral coherence elements (shown as 1102) generated from three coherence analyses according to the method of FIG. 9A. In FIG. 11, the coherence analysis is performed between channels X and Y, channels X and Z, and channels Y and Z for a cardiac signal data set. The x-axis shows time (in seconds), and the y-axis shows frequency (Hz). The binarized wavelet spectral coherence elements 1102 are generated, in FIG. 11, via a static threshold of 0.95 (where 1.00 indicates that power at that frequency and time is identical across both channels). In FIG. 11, it can be observed that there exists significant coherence between channels X and Z. Further, it can be observed that the coherence between channels X and Z is typically maximal in the QRS and drops during the T and the TP isoelectric period. In contrast, the other pairs of channels are much less frequently coherent above the static threshold of 0.95.

### Example #2—Wavelet-Based Features

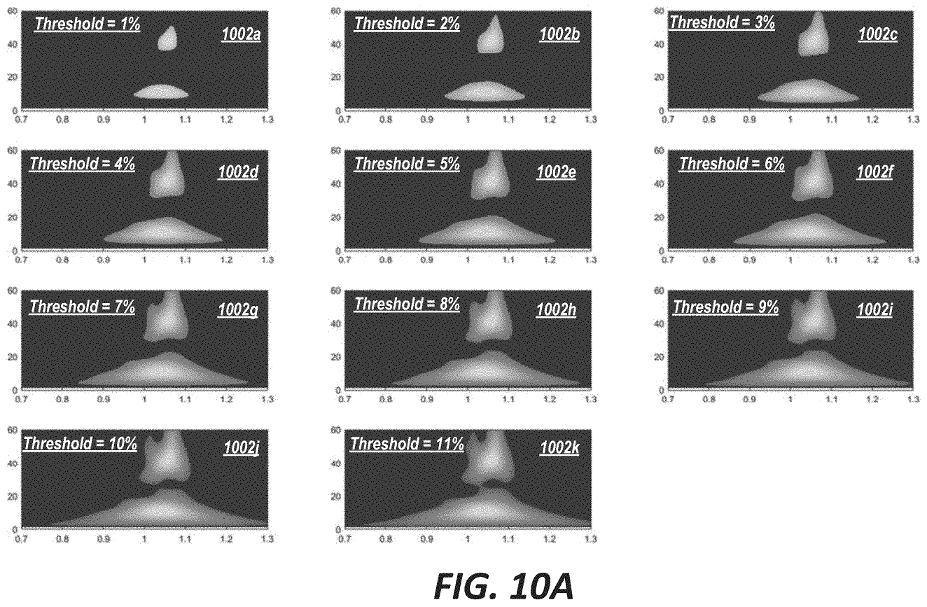

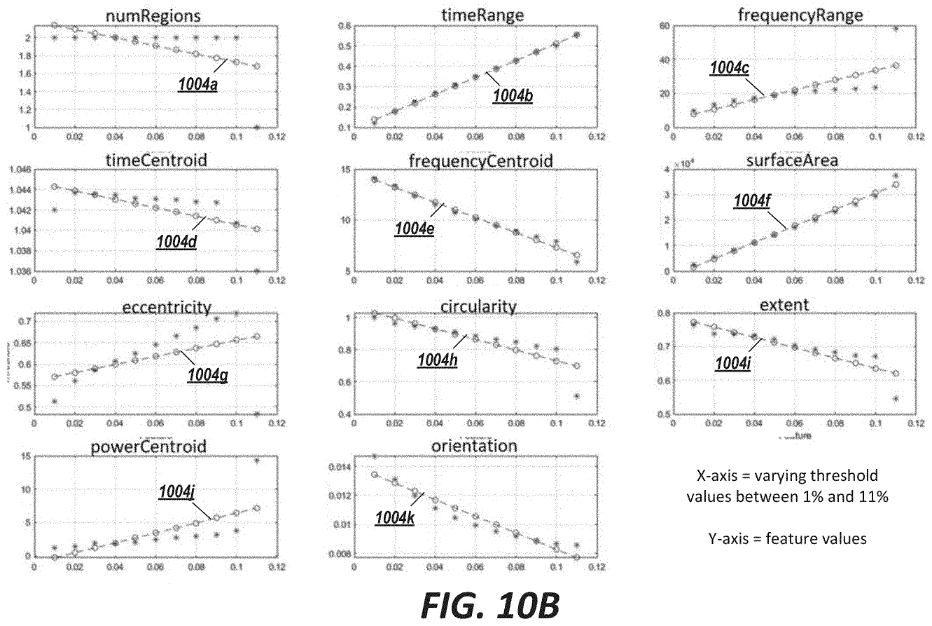

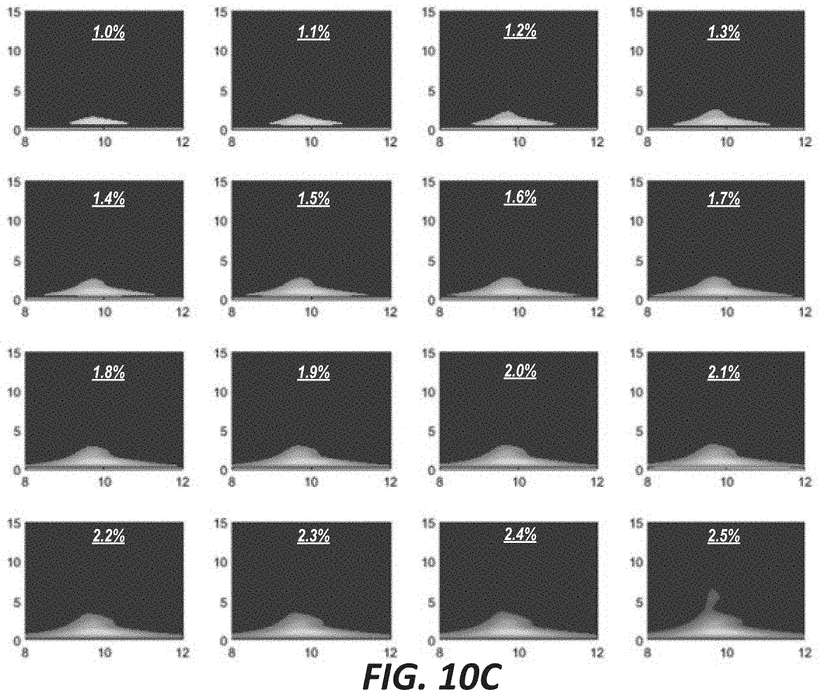

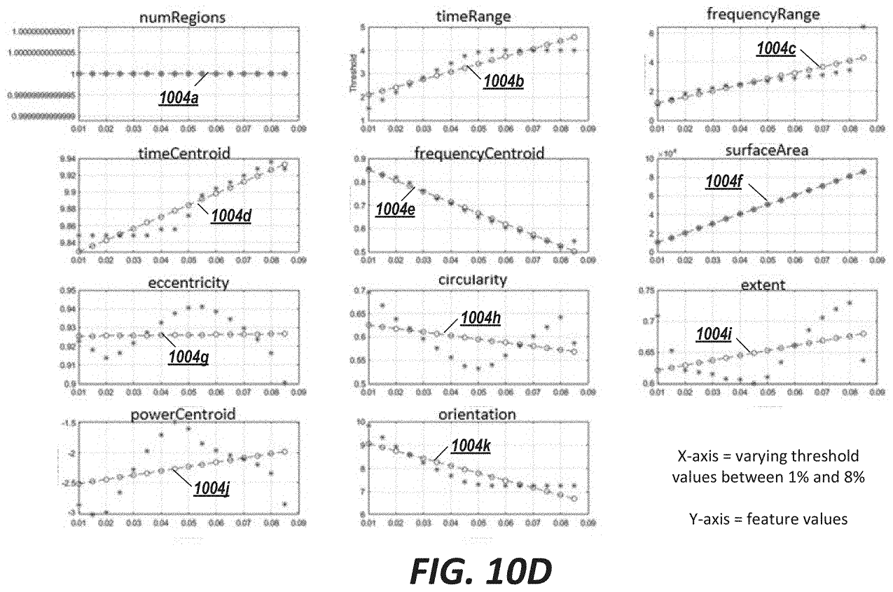

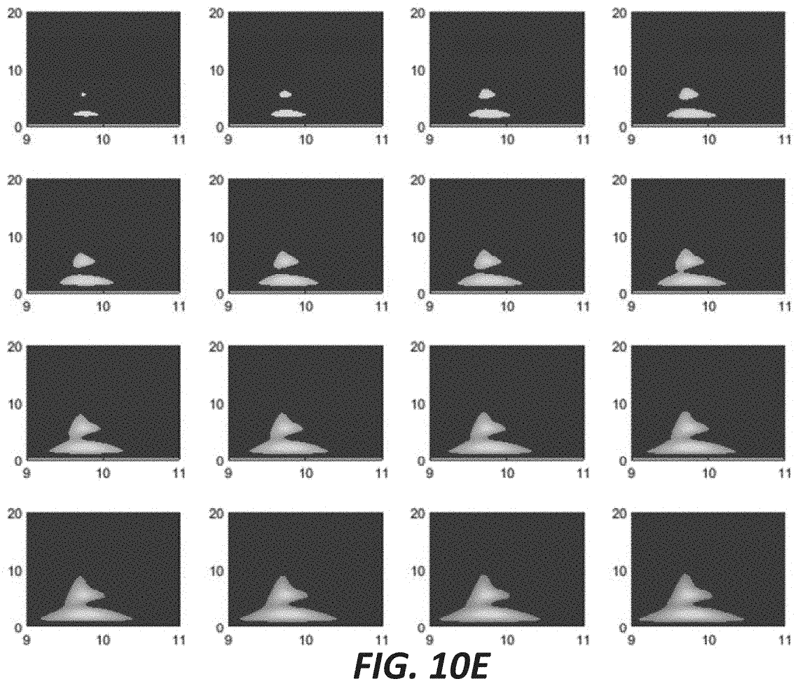

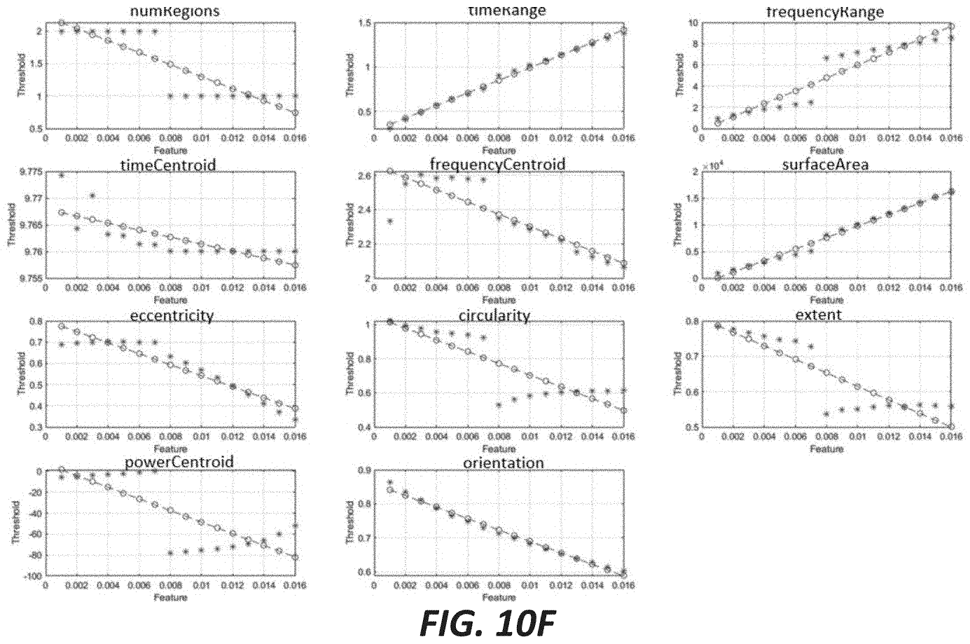

FIG. 4B illustrates, as the second of three example feature or parameter categories, an example wavelet trend feature computation module 400b configured to extract high spectral energy characteristics of a waveform region of interest in a given biophysical signal. FIGS. 10A-10F show example methods of the wavelet feature module of FIG. 4B to compute wavelet-based features from trends established in multiple binarized spectral images of the spectral wavelet model of a cardiac signal, a photoplethysmographic signal, and a velocityplethysmogram signal, in accordance with an illustrative embodiment.

Due to the potential sensitivity of wavelet features or parameters of Module 400a to the power threshold value, Module 400a may be complemented or supplemented with additional wavelet-based features or parameters of Modules 400b to mitigate such dependence. Module 400b may generate a spectral image or spectral data from a wavelet transformation of a given waveform region of interest (or used intermediate outputs from Module 400a) to which multiple threshold operations at different threshold values are performed to generate a corresponding number of binarized spectral images or data. Each binarized spectral image or data is evaluated for a given parameter (e.g., time range, frequency range, time centroid, surface area, eccentricity, circularity, extent, orientation, and/or power centroid) of the high spectral energy characteristics of a waveform region to which a trend line may be determined. This trend and sensitivity evaluation is also referred to as “decay” analysis.

FIG. 6B shows a method 600b to generate wavelet-based features or parameters, e.g., as performed by the wavelet trend feature computation module 400b of FIG. 4B, in accordance with an illustrative embodiment, which can be used wholly, or partially, to generate wavelet-based features or parameters and its outputs to be used in machine-learned classifier to determine a metric associated with a physiological system of a subject under study. To determine the wavelet-based features or parameters, Module 400b is configured, in some embodiments, to (i) pre-process (602) the acquired biophysical signal, (ii) isolate (604) waveform regions of interest of a given signal, (iii) generate (606) a wavelet-power spectrum (or coherence model) of the isolated regions as a spectral image or data. Rather than applying a single threshold, Module 400b is configured to apply multiple threshold operations at different threshold values to generate a corresponding number of binarized spectral images or data.

Tables 4A and 4B each shows a summarized set of 99 wavelet-based features (“parameters”) computed from a threshold sensitivity analysis of the high-power content spectral content of the waveform signal regions of interests for a given signal channel of a biopotential signal (referred to as “Signal Channel”). In Table 4A, the wavelet-based features are shown generated from three potential waveform regions (shown as “Signal Region”), namely the atrial depolarization (P-wave) regions, ventricular depolarization (QRS) wave regions, and ventricular repolarization (T-wave) regions, and for each of the acquired channels (e.g., X, Y, and Z channels). The parameter may be computed across many cycles and quantified as the interquartile range (“IQR”) or as the median to provide a total of 198 features.

In Table 4A, the geometric or properties associated with “Decay_TimeRange,” “Decay_FrequencyRange,” “Decay_TimeCentroid,” FrequencyCentroid,” “Decay_Eccentricity,” “Decay_Circularity,” “Decay_Extent,” “Decay_Orientation,” and “Decay_PowerCentroid” for each of the waveform signal regions and signal channels have been experimentally determined to have significant utility in the assessment of the presence or non-presence of at least one cardiac disease or condition—specifically, the determination of presence or non-presence of elevated LVEDP. It also has been observed through experimentation that Decay_TimeRange,” “Decay_TimeCentroid,” “Decay_SurfaceArea,” “Decay_Eccentricity,” “Decay_Circularity,” “Decay_Extent,” and “Decay_Orientation” parameters have significant utility in the assessment of the presence or non-presence of coronary artery disease. The list of the specific features determined to have significant utility in the assessment of the presence or non-presence of abnormal or elevated LVEDP and the presence or non-presence of significant CAD is provided in Table 8B and Table 9B, respectively.

TABLE 4A

| Signal Region | | Signal Channel | |
|---|---|---|---|
| to which Features | | to which features | Distribution |
| are extracted | Feature Name | are extracted | Summary |
| wtPwave | Decay_TimeRange*^{,} ** | X | Iqr |
| wtQRSwave | Decay_FrequencyRange* | Y | Median |
| wtTwave | Decay_TimeCentroid*^{,} ** | Z | |
| | Decay_FrequencyCentroid* | | |
| | Decay_SurfaceArea** | | |
| | Decay_Eccentricity*^{,} ** | | |
| | Decay_Circularity*^{,} ** | | |
| | Decay_Extent*^{,} ** | | |
| | Decay_Orientation*^{,} ** | | |
| | Decay_PowerCentroid* | | |
| | Decay_Numregions* | | |

Table 4B shows the 11 features of Table 1 computed for three waveform signal regions (e.g., maximum peak regions, minimum peak regions, and base pulse regions) of photoplethysmographic signal and/or a corresponding velocityplethysmogram signal (e.g., PPG IR channel, PPG red channel, VPG IR channel, and VPG red channel) to provide a total of 132 features. The features may be generated across multiple cycles, which can be expressed in interquartile range or median distribution to provide a total number of 264 features.

TABLE 4B

| Signal Region | | Signal Channel | |
|---|---|---|---|
| to which Features | | to which features | Distribution |
| are extracted | Feature Name | are extracted | Summary |
| Maximum Peak | Decay_TimeRange | PPG_IR | Iqr |
| Minimum peak region | Decay_FrequencyRange | PPG_red | median |
| Base pulse region | Decay_TimeCentroid | VPG_IR | |
| | Decay_FrequencyCentroid | VPG_red | |
| | Decay_SurfaceArea | | |
| | Decay_Eccentricity | | |
| | Decay_Circularity | | |
| | Decay_Extent | | |
| | Decay_Orientation | | |
| | Decay_PowerCentroid | | |
| | Decay_Numregions | | |

In an example, FIG. 10A shows a set of binarized spectral images generated from different threshold values for one waveform region (namely, P-wave region) in one channel of an acquired cardiac signal. In FIG. 10A, variations of power suppression are shown based on the power threshold ranging from 1% to 11% (shown as images 1002a-1002k).

FIG. 10B shows the 11 parameters of Table 1 for different levels of threshold values (from 1% to 11%) and a corresponding trend line derived from that the respective data. In FIG. 10B, a fit function (shown as 1004a-1004k) is applied to which a parameter (e.g., the slope) associated with the fit is used as a wavelet feature or parameter. In FIG. 10B, the y-axis of each plot shows the parameter value, and the x-axis shows the threshold value associated with that parameter value.

FIGS. 10C and 10D shown similar computation of wavelet-based features or parameters of a photoplethysmographic signal. In FIG. 10C, threshold values are shown varied between 1% and 2.5%. In FIG. 10D, the 11 parameters of Table 1 are shown for different levels of threshold values (from 1% to 8%) for a photoplethysmographic signal.

Wavelet Distribution Features—Example #3

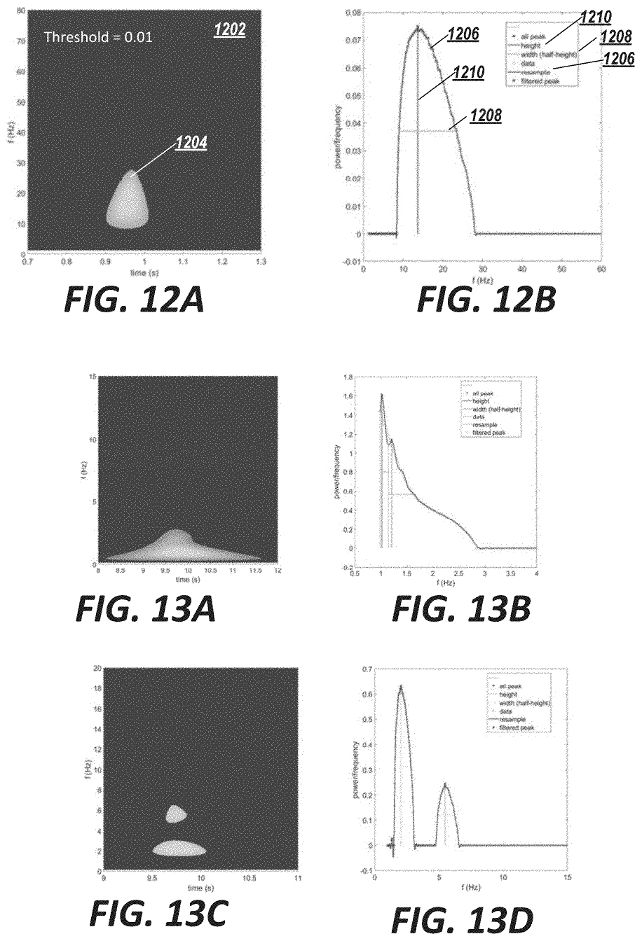

FIG. 5 illustrates, as the third of the three example feature or parameter categories, an example wavelet distribution feature computation module 500 configured to assess, using statistical operations, the distribution of the power in time and frequency of a waveform region of interest in a given biophysical signal. FIGS. 12A-12B, 13A-13D, and 14A-14D show example methods of the wavelet distribution feature module of FIG. 5 to generate wavelet distribution features in accordance with an illustrative embodiment.

Module 500 is configured to calculate the power distribution for the high-power region of the power spectrum for the P-wave, QRS complex, and T-wave separately filtered by using the power percentile threshold. The distribution analysis is performed over the frequencies in the range of 1-60 Hz to focus on the analysis of the majority of the frequency component existing in the ventricular depolarization and repolarization waves.

The distribution may include the quantile-quantile probability assessed between (i) a distribution function determined within the wavelet-based models and (ii) a base distribution function (e.g., uniform or normal distribution). The distribution may be based on the power density function or cumulative density function.

Table 5 shows an example set of 25 extractable wavelet-based features or parameters of the power distribution for the high-power region of the power spectrum of waveform regions of interest. The first 13 features are calculated from the power spectral density distribution.

TABLE 5

| Feature Name | Feature Description |
|---|---|
| Dist_sumPSD | The sum of the power spectral density function of a high- |
| | spectral region of the waveform of interest in a biophysical |
| | signal, the power spectral density determined from the high |
| | spectral energy region in a binarized spectral image or data. |
| Dist_meanPSD | The mean of the power spectral density function of a high- |
| | spectral region of the waveform of interest in a biophysical |
| | signal, the power spectral density determined from the high |
| | spectral energy region in a binarized spectral image or data. |
| Dist_medianPSD | The median of the power spectral density function of a high- |
| | spectral region of the waveform of interest in a biophysical |
| | signal, the power spectral density determined from the high |
| | spectral energy region in a binarized spectral image or data. |
| Dist_stdPSD | The standard deviation of the power spectral density function |
| | of a high-spectral region of the waveform of interest in a |
| | biophysical signal, the power spectral density determined |
| | from the high spectral energy region in a binarized spectral |
| | image or data. |
| Dist_skewPSD | The skewness of the power spectral density function of a |
| | high-spectral region of the waveform of interest in a |
| | biophysical signal, the power spectral density determined |
| | from the high spectral energy region in a binarized spectral |
| | image or data. |
| Dist_kurtPSD | The kurtosis of the power spectral density function of a high- |
| | spectral region of the waveform of interest in a biophysical |
| | signal, the power spectral density determined from the high |
| | spectral energy region in a binarized spectral image or data. |
| Dist_Entropy | The entropy (Eᵢ) of the power spectral density function of a |
| | high-spectral region of the waveform of interest in a |
| | biophysical signal, the entropy determined as follows, where |
| | P_{ω} is the power spectral density for a signal i. |
| | ${E}_{i} = - {\sum }_{ω = 0}^{ω = {N}_{bin}} {P}_{ω} ( {c}_{i} ) log {P}_{ω} ( {c}_{i} ) i = x , y , z$ |
| Dist_peakWidthPSD | The width of an assessed largest peak (at a halfway height of |
| | that peak) in a power spectral density function of a high- |
| | spectral region of the waveform of interest in a biophysical |
| | signal, the power spectral density determined from the high |
| | spectral energy region in a binarized spectral image or data. |
| Dist_numpeakPSD | A number of assessed peaks in a power spectral density |
| | function of a high-spectral region of the waveform of interest |
| | in a biophysical signal, the power spectral density determined |
| | of a thresholded object in a binarized spectral image. |
| Dist_peakRatioPSD | The mean of the ratio of the consecutive spectral peaks in the |
| | power spectral density function of a high-spectral region of |
| | the waveform of interest in a biophysical signal, the power |
| | spectral density determined of a thresholded object in a |
| | binarized spectral image. The mean is set to a default/null |
| | value if there is only a single peak. |
| Dist_fpeakRatioPSD | The mean of the ratio of the consecutive spectral peak |
| | frequencies in the power spectral density function of a high- |
| | spectral region of the waveform of interest in a biophysical |
| | signal, the power spectral density determined of a thresholded |
| | object in a binarized spectral image. The mean is set to a |
| | default/null value if there is only a single peak. |
| Dist_peakDispRate | The peak dispersion rate (with respect to frequency) is the |
| | mean of the difference in consecutive peak dispersion |
| | normalized by the maximum value of the peak dispersion. |
| | The peak dispersion rate is set to a default/null value if there |
| | is only one spectral peak detected. |
| Dist_peakDispPSD | Peak dispersion is the ratio of spectral peak amplitude to its |
| | corresponding spectral peak width in a power spectral density |
| | function of a high-spectral region of the waveform of interest |
| | in a biophysical signal, the power spectral density determined |
| | of a thresholded object in a binarized spectral image. |
| Dist_qqslop uniform | The slope of a regression line (qqslop) applied to a quantile- |
| | quantile probability plot of the quantiles of the assessed |
| | power spectral density (PSD) versus base quantile values |
| | from a uniform power spectral density (PSD). The slopes |
| | are calculated for each region across the multiple cycles, and |
| | the median is selected. |
| Dist_qqsse uniform | The standard error (qqsse) of an estimation for a best linear |
| | fit function applied to a quantile-quantile probability plot of |
| | the quantiles of the assessed power spectral density (PSD) |
| | versus base quantile values from a uniform power spectral |
| | density (PSD). |
| Dist_qqr2 uniform | The adjusted R-squared (qqr2) of a fit function applied to a |
| | quantile-quantile probability plot of the quantiles of the |
| | assessed power spectral density (PSD) versus base quantile |
| | values from a uniform power spectral density (PSD). |
| Dist_qqslop normal | The slope of a regression line (qqslop) applied to a quantile- |
| | quantile probability plot of the quantiles of the assessed |
| | power spectral density (PSD) versus base quantile values |
| | from a normal (Gaussian) power spectral density (PSD). |
| Dist_qqsse normal | The standard error (qqsse) of an estimation for a best linear |
| | fit function applied to a quantile-quantile probability plot of |
| | the quantiles of the assessed power spectral density (PSD) |
| | versus base quantile values from a normal (Gaussian) power |
| | spectral density (PSD). |
| Dist_qqr2 normal | The adjusted R-squared (qqr2) of a fit function applied to a |
| | quantile-quantile probability plot of the quantiles of the |
| | assessed power spectral density (PSD) versus base quantile |
| | values from a normal (Gaussian) power spectral density |
| | (PSD). |
| Dist_cdfNormal_L 1norm | The sum of L 1 distance of the absolute difference between |
| | (i) a cumulative density distribution (CDD) of the signal |
| | PSD and (ii) a normal (Gaussian) cumulative density |
| | distribution (CDD). |
| Dist_cdfNormal_std | The standard deviation of the L 1 distance of the absolute |
| | difference between (i) a cumulative density distribution |
| | (CDD) of the signal PSD and (ii) a normal (Gaussian) |
| | cumulative density distribution (CDD). |
| Dist_pdftkernel_L 1norm | The sum of L 1 distance between (i) a probability density |
| | distribution (PDD) of the signal PSD and (ii) a kernel |
| | density estimator (KDE) fitted to the PDD. |
| Dist_pdfkernel_std | The standard deviation of value of kernel density estimator |
| | (KDE) fitted to a probability density distribution (PDD) of a |
| | power spectral density (PSD) of a thresholded object in a |
| | binarized spectral image. |
| Dist_pdftkernel_numpeak | The number of peaks of kernel density estimator (KDE) |
| | fitted to a probability density distribution (PDD) of a power |
| | spectral density (PSD) of a thresholded object in a binarized |
| | spectral image. |
| Dist_pdftkernel_dpowerPeak | The peak amplitude value of kernel density estimator (KDE) |
| | fitted to a probability density distribution (PDD) of a power |
| | spectral density (PSD) of a thresholded object in a binarized |
| | spectral image. |

FIG. 6C shows a method 600c to generate wavelet-based features or parameters, e.g., as performed by the wavelet distribution feature computation module 500 of FIG. 5, in accordance with an illustrative embodiment, which can be used wholly, or partially, to generate wavelet-based features or parameters and its outputs to be used in machine-learned classifier to determine a metric associated with a physiological system of a subject under study. To determine the features of Table 5, Module 500 is configured, in some embodiments, to (i) pre-process (602) the acquired biophysical signal, (ii) isolate (604) waveform regions of interest of a given signal, (iii) generate (606) a wavelet-power spectrum (or coherence model) of the isolated regions as a spectral image or data, (iv) generate (612) a binarized spectral image or data from the spectral image or data via a threshold operator (v) determine (614) power spectral density (PSD) or cumulative density function (CDF) of the binarized image or data, and (vi) determine (616) feature from aspects of the power spectral density (PSD) or cumulative density function (CDF).

In FIG. 6C, the method 600c includes pre-processing (602) the acquired biophysical signal (e.g., sub-signaling, down-sampling, and baseline removal) as described in relation to FIG. 6A. The method 600c then includes detecting (604), e.g., using wavelet transform, one or more regions of spectral interest in the biophysical signals as described in relation to FIG. 6A. The method 600c then includes generating (606), using wavelet decomposition/transform operation, a wavelet-power spectrum model of the isolated regions as a spectral image or 2D/3D spectral data, e.g., as described in relation to FIG. 6A.

The method 600c then includes determining (612) the high-power region of a generated wavelet spectral image. For cardiac signals, for example, the high-power region of the P-wave, QRS complex, and T-wave may be assessed, e.g., using filters having a power percentile threshold of 0.01, 0.01, and 0.025, respectively. The distribution analysis is limited to the frequencies in the range of 1-60 Hz, which contains the majority of the frequency component existing in the ventricular depolarization and repolarization waves. For photoplethysmographic signal, the wavelet power spectrum of the PPG and VPG Red are filtered by using the 4% and 0.3% highest power percentile (corresponding to the threshold of 0.04, 0.003), respectively. The distribution analysis for PPG and VPG is limited to frequencies below 10 Hz and 15 Hz, respectively.

The method 600c then includes determining (614) distribution of the high-power region, such as the power spectrum density or the cumulative distribution function. In some embodiments, the power spectrum density (PSD) is obtained by integrating the power (e.g., the z-axis of the image) over the spectrum time (e.g., the x-axis of the image) at each specified frequency.

The method 600c then includes determining (616) features and associated values from aspects of the generated distribution per Tables 6A and 6B provided below.

Cardiac Wavelet Distribution Features. Table 6A shows a summarized set of 213 features directed to the 25 distribution parameters of Table 5. In Table 6A, 21 features may be determined for any one of three example waveform regions (e.g., atrial depolarization (P-wave) regions, ventricular depolarization (QRS) wave regions, and ventricular repolarization (T-wave) regions) of a given cardiac signal (e.g., channels X, Y, and Z) while 4 of the features may be determined for two of the regions. The wavelet-based feature or parameter may be computed from spectral images generated across many cardiac cycles. To compress the computed feature values down to a summary feature (e.g., suitable for ML), Module 500 may employ the median in the distribution of feature values.

It has been observed that 14 of the features (“Dist_medianPSD,” “Dist_stdPSD,” “Dist_skewPSD,” “Dist_entropy,” “Dist_peakWidthPSD,” “Dist_peakDispPSD,” “Dist_qqslop uniform,” “Dist_qqr2 uniform”, “Dist_qqsse normal,” “Dist_qqr2 normal”, “Dist_cdfNormal_L1norm”, “Dist_cdfNormal_std,” “Dist_pdftkernel_L1norm”, and “Dist_pdfkernal_std”) among various waveform signal regions and signal channels have been experimentally determined to have significant utility in the assessment of the presence or non-presence of at least one cardiac disease or condition—specifically, the determination of presence or non-presence of elevated LVEDP. It also has been observed through experimentation that “medianPSD,” “stdPSD,” “kurtPSD,” “entropy,” “peakWidthPSD,” “qqslop_uniform,” and “qqr2_uniform,” “qqslop_normal,” “qqsse_normal,” “qqr2_normal,” “dfNormal_L1norm,” and “pdfKernal_std” parameters have significant utility in the assessment of the presence or non-presence of coronary artery disease. The list of the specific wavelet-based features or parameters determined to have significant utility in the assessment of the presence or non-presence of abnormal or elevated LVEDP and the presence or non-presence of significant CAD is provided in Table 8C and Table 9C, respectively.

TABLE 6A

| Signal Region | | Signal Channel | |
|---|---|---|---|
| to which Features | | to which features | Distribution |
| are extracted | Feature Name | are extracted | Summary |
| wtPwave | Dist_sumPSD | X | median |
| wtQRSwave | Dist_meanPSD | Y | |
| wtTwave | Dist_medianPSD*^{,} ** | Z | |
| | Dist_stdPSD*^{,} ** | | |
| | Dist_skewPSD* | | |
| | Dist_kurtPSD** | | |
| | Dist_entropy*^{,} ** | | |
| | Dist_peakWidthPSD*^{,} ** | | |
| | Dist_numpeakPSD* | | |
| | Dist_peakRatioPSD* | | |
| | Dist_fpeakRatioPSD* | | |
| | Dist_peakDispRate* | | |
| | Dist_peakDispPSD* | | |
| | Dist_qqslop uniform*^{,} ** | | |
| | Dist_qqsse uniform | | |
| | Dist_qqr2 uniform*^{,} ** | | |
| | Dist_qqslop normal** | | |
| | Dist_qqsse normal*^{,} ** | | |
| | Dist_qqr2 normal*^{,} ** | | |
| | Dist_cdfNormal_L1norm*^{,} ** | | |
| | Dist_cdfNormal_std*^{,} ** | | |
| | Dist_pdftkernel_L1norm* | | |
| | Dist_pdfkernal_std*^{,} ** | | |
| | Dist_pdftkernal_numpeak | | |
| | Dist_pdftkernal_dpowerPeak | | |

PPG/VPG Wavelet Distribution Features. Table 6B shows a summarized set of 50 features directed to the 25 distribution parameters of Table 5. In Table 6B, the 25 features may be determined for each cycle per signal (e.g., VPG and PPG) and compressed by their median across the cycles. It also has been observed through experimentation that “sumPSD,” “stdPSD,” “skewPSD,” “kurtPSD,” “entropy,” “peakWidthPSD,” “peakRatioPSD,” “numpeakPSD,” peakRatioPSD,” “fpeakRatioPSD,” “peakDispRate,” “qqslop_uniform,” “qqsse_normal,” “qqslop_normal,” “Pdftkernal_L1_norm,” “Pdftkernal_std,” “powerpeak,” “peakDispPSD,” and “Pdftkernal_numpeak” parameters have significant utility in the assessment of the presence or non-presence of coronary artery disease. The list of the specific wavelet-based features or parameters determined to have significant utility in the assessment of the presence or non-presence of significant CAD is provided in Table 9C.

TABLE 6B

| | Signal channel to which |
|---|---|
| Feature Name | feature are extracted |
| Dist_sumPSD | PPG_red |
| Dist_meanPSD** | VPG_red |
| Dist_medianPSD | |
| Dist_stdPSD** | |
| Dist_skewPSD** | |
| Dist_kurtPSD** | |
| Dist_Entropy** | |
| Dist_peakWidthPSD** | |
| Dist_numpeakPSD** | |
| Dist_peakRatioPSD** | |
| Dist_fpeakRatioPSD** | |
| Dist_peakDispRate** | |
| Dist_qqslop uniform** | |
| Dist_qqsse uniform** | |
| Dist_qqr2 uniform** | |
| Dist_qqslop normal** | |
| Dist_qqsse normal | |
| Dist_qqr2 normal | |
| Dist_cdfNormal_l1norm | |
| Dist_cdfNormal_std | |
| Dist_Pdftkernal L1norm** | |
| Dist_Pdftkernal_std** | |
| Dist_Pdftkernal_numpeak** | |
| Dist_Pdftkernal_powerpeak** | |
| Dist_peakDispPSD** | |

PSD Distribution Features. FIGS. 12A, 13A, and 13C each shows a wavelet spectral image 1202 comprising an assessed high-power region (shown as 1204) of a wavelet spectrum of a cardiac signal, a photoplethysmographic signal, and a velocityplethysmogram signal, respectively. FIGS. 12B, 13B, and 13D show assessed power density distribution characteristic profiles (1206) of the high-power region of FIGS. 12A, 13A, and 13D, respectively. Example extracted parameters (in FIGS. 12A, 13B, and 13D) include the width (1208) and the height (1210) of the peaks that may be used to assess the various features in this set.

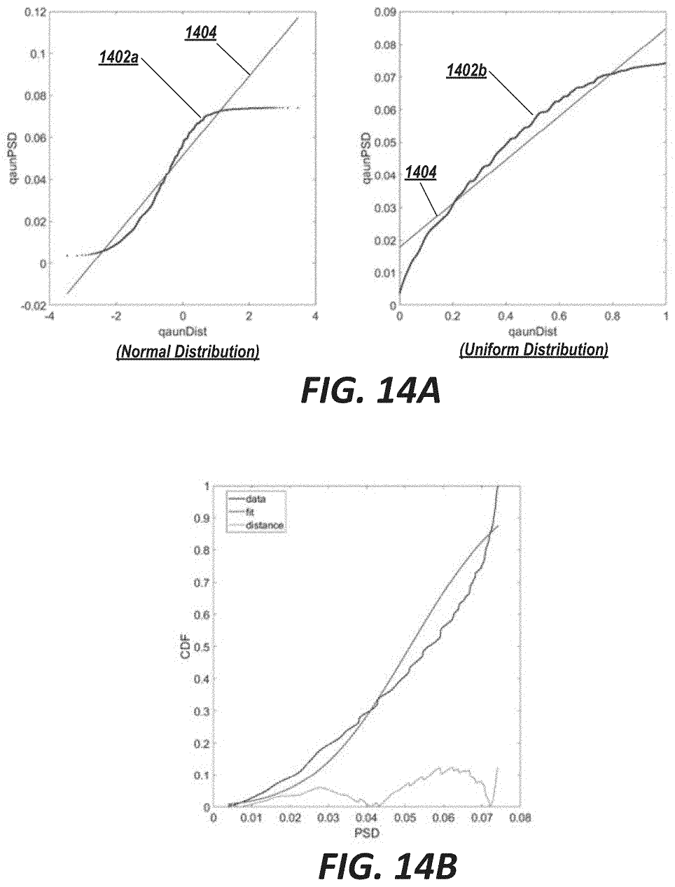

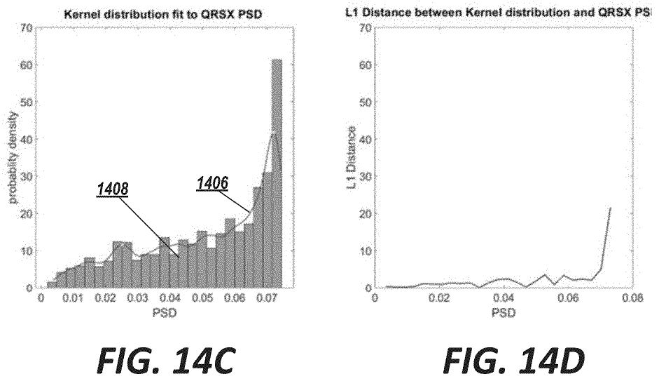

Power Spectral Density (PSD) Quantile-Quantile Probability Features. FIG. 14A shows an example quantile-quantile probability plot (shown as 1402a and 1402b) of the quantiles of a power spectral density function (y-axis) of the high-power spectral content of a waveform plotted against a theoretical quantile value (e.g., a Gaussian normal distribution and a uniform distribution) (x-axis). If the PSD data comes from the same theoretical distribution, then the data plot appears linear (shown as line 1404) with the slope indicating the scale. In the analysis, deviations from the linearity in the quantile-quantile plot may be captured by calculating the adjusted R-squared (e.g., “qqr2” features of Tables 6A or 6B) or the standard error (e.g., “qqsse” features of Tables 6A or 6B) of estimation for the best linear fit against the data in the quantile-quantile plot. The slope of the regression line may be outputted as a feature (e.g., “qqslop” feature of Tables 6A or 6B). The features are calculated for each cardiac waveform (e.g., atrial depolarization (P-wave) regions, ventricular depolarization (QRS) wave regions, and ventricular repolarization (T-wave) regions) across several cycles and compressed by their median.

Cumulative Density Distribution (CDD) Features. FIG. 14B shows an example cumulative density distribution (CDD) of the power spectral density (PSD) function (y-axis) plotted against a Gaussian normal CDD function (x-axis). The “Dist_cdfNormal_11norm” features is extracted from the absolute difference between the CDDs as the L1 distance. The “Dist_cdfNormal_std” feature from CDD is the sum of L1 distance and the standard deviation of L1 distance. FIG. 14C shows an example kernel density estimator (KDE) (1406) fitted to the power density distribution (PDD) (1408) of the power spectral density (PSD) of the high-power spectral region of the binarized spectral image or data. FIG. 14D shows an example L1 distance determined between the regression line of FIG. 14C and the data, which can be used to provide an indication of the goodness of the estimation.

Waveform Region Isolation Operation

Cardiac signals. FIGS. 8A-8C show waveform region isolation operations for a cardiac signal. FIG. 8A shows a diagram of a method 604a to delineate one or more of the waveforms associated with the ventricular depolarization (QRS), atrial depolarization (P-wave), and ventricular repolarization (T-wave).

The delineation operation 604a, in the example of FIG. 8A, includes transforming (802) the channel signals of the acquired cardiac signal data set to a single time-series ŷ per ŷ=\sqrt{y_{X}²+y_{Y}²+y_{Z}²}. The delineation operation 604a then includes detecting (804) peaks (corresponding to the ventricular depolarization (QRS peak)) in each cycle of the transformed time series (ŷ), e.g., via a local maxima detector (e.g., the findpeak function in Matlab).

FIG. 8B shows a delineation operation 806 (shown as 806a) to determine the ventricular depolarization (VD) onset and ventricular depolarization (VD) offset region of a transformed signal in accordance with an illustrative embodiment. The delineation operation 806a includes decomposing (810) channels of the cardiac signal using a wavelet transform., e.g., a continuous 1-D Morlet wavelet, Gaussian, Mexican Hat, Spline, and Mayer wavelet (e.g., as a mother wavelet). The delineation operation 806a then includes generating (812) a time-series signal from a bandpass filtered wavelet spectrum (e.g., between 40-60 Hz). The delineation operation 806a then includes determining (814) indices in the reconstructed signal, e.g., having cumulative power that falls below a dynamic threshold (e.g., top 25 percentile of the cumulative power) the first time before and after each detected ventricular depolarization (VD) peak. An offset-onset correction may be performed using the derivative of the signals to remove any falsely detected fiducial points. The three-channel correction may be applied to the detected ventricular depolarization onset and offset to eliminate any false landmarks that assign the same time index for the onset and offsets to all three channels.

The delineation operation 604a then includes segmenting/isolating (808) waveforms associated with ventricular repolarization and atrial depolarization in the transformed signal (806). In some embodiments, the ventricular repolarization and atrial depolarization segment (also referred to as T-P segment) is isolated by segmenting the signal between the QRS_{off,i} and the next consecutive cycle QRS_{onset,i+1}. Having T-P segment isolated, ⅔ of the TP segment may be assigned to the waveform region associated with ventricular repolarization (T-wave) and the remaining ⅓ to atrial depolarization (P-wave). Other segmentation may be used, for example, that described in D. B. Dubin, Rapid Interpretation of EKG's: An interactive course, 6th ed. tampa: Cover Pub, 2000.

FIG. 8C shows an example cardiac signal with delineated fiduciary or landmarks of interest using the method 604a described in FIG. 8A in accordance with an illustrative embodiment. In FIG. 8C, a downsampled cardiac signal (818), a bandpass filtered representation of the transformed signal 9 (820) and detected ventricular depolarization onsets and offsets (822 and 824, respectively) are shown for each of three example cardiac signals. FIG. 8D shows an example cardiac signal data set comprising three channels (824, 826, and 828). FIG. 8D further shows the isolated cardiac waves for the QRS complex (830a, 830b, 830c), the T-wave (832a, 832b, 832c), and the P-wave (834a, 834b, 834c) for each channel of the cardiac signal data set that are extracted from each of the cycles. A random beat (836) is shown for reference. In 830a-830c, 832a-832c, and 834a-834c, the multiple cycles (836) are shown along with a randomly selected beat (838) for reference.

Photoplethysmographic Signals. FIGS. 8E-8F show waveform region isolation operations (e.g., a Hemodynamic Delineator) for the photoplethysmographic and velocityplethysmogram signals. FIGS. 8E and 8F show an example method 604b and 604c, respectively, to detect and isolate regions of spectral interest of the photoplethysmographic and velocityplethysmogram signals. In FIG. 8E, the Hemodynamic Delineator operation is shown to detect the PPG base landmarks, the PPG systolic peak landmarks, and the diastolic peak landmarks. In FIG. 8F, the Hemodynamic Delineator operation is shown to detect the VPG peak landmarks, the PPG min landmarks, and the diastolic base pulse landmarks.

The Hemodynamic Delineator may first determine the PPG pulse base, which can then be used for the detection of other landmarks. To determine the PPG pulse base, in FIG. 8E, method 836 includes (i) inverting (848) the signal so the pulse bases are presented as peaks in the inverted time series, (ii) detecting (850) peaks, (iii) filtering (852) the detected peaks, and (iv) re-inverting (854) the signal again back to its original position. A peak (per operation 852) may be defined as having a minimum pulse of 125 ms (e.g., equal to 25% of the minimum beat duration, 500 ms at the heart rate of 120 bpm). The filter (operation 852) may use the criteria: (i) the minimum peak width (at the half-prominence) having less than the minimum pulse width of 125 ms, (ii) pulse base values should always be smaller than zero (since the DC component of the signal is removed), (iii) and pulse base value should be less than ten scaled median absolute deviations (MAD) away from the median of the detected pulse bases.

Following the PPG pulse base determination, systolic peak 838 may be determined. The peak may be defined as having a minimum pulse of 125 ms and filtered using the criteria: (i) the minimum peak width (at the half-prominence) being less than the minimum pulse width of 125 ms and (ii) systolic peak values being less than ten-scaled median absolute deviations (MAD) away from the median of the detected pulse bases. A maximum filter may be applied to detect the maximum values of the detected peaks within two consecutive pulse bases as the systolic peak for a corresponding cycle.

Following the PPG pulse base determination, a diastolic peak may be determined. The diastolic peak (operation 842) may be determined by (i) segmenting (858) the PPG signal using indexes of VPG min (discussed below) at cycle n and the consecutive PPG pulse base at cycle n+1, smoothing (860) the segmented PPG using a smoothing operator, determining (862) a VPG signal from the smoothed PPG signal, and detecting (862) peaks in the determined VPG signal. The smoothing operation (860) may employ a 20-datapoint Gaussian-weighted moving average filter as one example. The peak detection (864) may be configured to detect VPG peaks by application of a time constraint that constrains the local maxima to the first 50% of indexes of the segmented VPG. In some embodiments, a maximum filter is applied to the original PPG (non-smoothed) to search for local maxima around the detected diastolic peak in the smoothed PPG to determine the diastolic peak.

To determine the VPG min, the PPG signal may be segmented using the indexes of PPG systolic peak at cycle n and the consecutive PPG pulse base at cycle n+1. The segmented PPG may be smoothened, e.g., with a 20-datapoint Gaussian-weighted moving average filter. A VPG signal may be derived from the smoothed PPG, and the peaks for the inverted VPG are detected using a peak finder operator. The detected peaks may be filtered by applying a time constrain that the VPG min should occur at the first 30% of indexes of the segmented VPG. A maximum filter is applied to the original VPG (non-smoothed) that searches for local minima around the detected VPG min in the smoothed VPG.

FIG. 8F shows an example velocityplethysmogram waveform segmentation operation 604c to detect the VPG peak landmarks (operations 842), the PPG min landmarks (operations 844), and the diastolic base pulse landmarks (operations 844).

To find a VPG peak, method 842 includes (i) segmenting (866) indexes from a photoplethysmographic signal corresponding to a monotonically increasing segment in the photoplethysmographic signal, also referred to as PPG raise-segments, (ii) detecting (868) VPG peaks using a peak finder operator, and (iii) filtering (870) the detected peaks and applying a maximum filter to identify the maximum value of the detected peaks within the PPG raise-segment as the VPG peak for the corresponding cycle. The PPG raise-segments (per operation 866) may be identified as the data points between the PPG pulse base at cycle n and a consecutive PPG systolic peak at cycle n+1. To detect (868) the VPG peaks, a peak finder operator may be used, e.g., configured with a minimum pulse width of 25% of the median of the PPG raise-duration (the time in ms associated with the PPG raise-segment) across the VPG signal. To filter the detected peaks, the filter may be applied with the criteria: (i) the minimum peak width (at the half-prominence) be less than the VPG minimum pulse width and (ii) VPG peak values be less than ten scaled MAD away from the median of the detected peaks.

To determine the VPG min, method 844 includes (i) segmenting (872) the PPG signal using the indexes of PPG systolic peak at cycle n and the consecutive PPG pulse base at cycle n+1, (ii) smoothing (874) the segmented PPG, (iii) determining (876) an inverted VPG signal from the smoothed PPG and detect peaks of the inverted VPG, and (iv) applying (878) a maximum filter to the original VPG (non-smoothed) to search for a local minima around the detected VPG min in the smoothed VPG. To smooth the segmented PPG, a 20-datapoint Gaussian-weighted moving average filter may be used as one example. To detect the peaks (876), a filter may be used that is configured to filter for detected peaks having a time constraint that the VPG min occurs at the first 30% of indexes of the segmented VPG. The method 844 may.

To determine the VPG base, method 846 include (i) correcting (880) a baseline from the VPG signal that may occur during the numerical derivatization of PPG and (ii) determining (882) the VPG bases through zero-crossing the VPG where VPG bases less than four-scaled median absolute deviations (MAD) away from the median of the detected bases are removed. The baseline correction (880) may be performed by percentile filtering the VPG to identify the data points with the values between the 20% and 50% percentile of the VPG signal and then subtracting the VPG signal from the mean of the percentile filtered VPG. The baseline correction 880 may segment the baseline-corrected VPG using the indexes of VPG min at cycle n and a consecutive VPG peak at cycle n+1.

### Experimental Results and Examples

Several development studies have been conducted to develop feature sets, and in turn, algorithms that can be used to estimate the presence or non-presence, severity, or localization of diseases, medical conditions, or an indication of either. In one study, algorithms were developed for the non-invasive assessment of abnormal or elevated LVEDP. As noted above, abnormal or elevated LVEDP is an indicator of heart failure in its various forms. In another development study, algorithms and features were developed for the non-invasive assessment of coronary artery disease.

As part of these two development studies, clinical data were collected from adult human patients using a biophysical signal capture system and according to protocols described in relation to FIG. 2. The subjects underwent cardiac catheterization (the current “gold standard” tests for CAD and abnormal LVEDP evaluation) following the signal acquisition, and the catheterization results were evaluated for CAD labels and elevated LVEDP values. The collected data were stratified into separate cohorts: one for feature/algorithm development and the other for their validation.

Within the feature development phases, features were developed, including the wavelet-based features, to extract characteristics in an analytical framework from biopotential signals (as an example of the cardiac signals discussed herein) and photo-absorption signals (as examples of the hemodynamic or photoplethysmographic discussed herein) that are intended to represent properties of the cardiovascular system. Corresponding classifiers were also developed using classifier models, linear models (e.g., Elastic Net), decision tree models (XGB Classifier, random forest models, etc.), support vector machine models, and neural network models to non-invasively estimate the presence of an elevated or abnormal LVEDP. Univariate feature selection assessments and cross-validation operations were performed to identify features for use in machine learning models (e.g., classifiers) for the specific disease indication of interest. Further description of the machine learning training and assessment are described in U.S. Provisional Patent Application No. 63/235,960, filed Aug. 23, 2021, entitled “Method and System to Non-Invasively Assess Elevated Left Ventricular End-Diastolic Pressure,” which is hereby incorporated by reference herein in its entirety.

The univariate feature selection assessments evaluated many scenarios, each defined by a negative and a positive dataset pair using t-test, mutual information, and AUC-ROC evaluation. The t-test is a statistical test that can determine if there is a difference between two sample means from two populations with unknown variances. Here, the t-tests were conducted against a null hypothesis that there is no difference between the means of the feature in these groups, e.g., normal LVEDP vs. elevated (for LVEDP algorithm development); CAD− vs. CAD+(for CAD algorithm development). A small p-value (e.g., ≤0.05) indicates strong evidence against the null hypothesis.

Mutual information (MI) operations were conducted to assess the dependence of elevated or abnormal LVEDP or significant coronary artery disease on certain features. An MI score greater than one indicates a higher dependency between the variables being evaluated. MI scores less than one indicate a lower dependency of such variables, and an MI score of zero indicates no such dependency.

A receiver operating characteristic curve, or ROC curve, illustrates the diagnostic ability of a binary classifier system as its discrimination threshold is varied. The ROC curve may be created by plotting the true positive rate (TPR) against the false positive rate (FPR) at various threshold settings. AUC-ROC quantifies the area under a receiver operating characteristic (ROC) curve—the larger this area, the more diagnostically useful the model is. The ROC, and AUC-ROC, value is considered statistically significant when the bottom end of the 95% confidence interval is greater than 0.50.

Table 7 shows an example list of the negative and a positive dataset pair used in the univariate feature selection assessments. Specifically, Table 7 shows positive datasets being defined as having an LVEDP measurement greater than 20 mmHg or 25 mmHg, and negative datasets were defined as having an LVEDP measurement less than 12 mmHg or belonging to a subject group determined to have normal LVEDP readings.

TABLE 7

| Negative Dataset | Positive Dataset |
|---|---|
| ≤12 (mmHg) | ≥20 (mmHg) |
| ≤12 (mmHg) | ≥25 (mmHg) |
| Normal LVEDP | ≥20 (mmHg) |
| Normal LVEDP | ≥25 (mmHg) |

Tables 8A, 8B, and 8C each shows a list of wavelet-based features having been determined to have utility in estimating the presence and non-presence of elevated LVEDP in an algorithm executing in a clinical evaluation system. The features of Tables 8A, 8B, and 8C and corresponding classifiers have been validated to have clinical performance comparable to the gold standard invasive method to measure elevated LVEDP.

TABLE 8A

| Feature_name | t-test | AUC | MI |
|---|---|---|---|
| wtPwave_circularity_X_iqr | 0.0041 | 0.5289 | n/s |
| wtPwave_circularity_Y_iqr | 0.0180 | 0.5199 | n/s |
| wtPwave_circularity_Y_median | 0.0075 | n/s | n/s |
| wtPwave_eccentricity_X_median | 0.0471 | n/s | n/s |
| wtPwave_eccentricity_Y_median | 0.0103 | n/s | n/s |
| wtPwave_frequencyCentroid_X_iqr | 0.0010 | 0.5304 | 1.1492 |
| wtPwave_powerCentroid_Z_median | 0.0492 | n/s | n/s |
| wtPwave_surfaceArea_X_iqr | 0.0014 | 0.5260 | 1.1946 |
| wtPwave_surfaceArea_X_median | 0.0025 | n/s | n/s |
| wtPwave_surfaceArea_Y_iqr | n/s | 0.5035 | n/s |
| wtPwave_timeCentroid_Y_iqr | n/s | 0.5015 | n/s |
| wtPwave_timeRange_X_iqr | 0.0386 | 0.5204 | n/s |
| wtPwave_timeRange_Y_iqr | 0.0133 | 0.5240 | n/s |
| wtPwave_timeRange_Z_median | n/s | 0.5204 | n/s |
| wtPwave_circularity_X_median¹ | 0.03125 | 0.5101 | n/s |
| wtPwave_timeCentroid_X_iqr¹ | n/s | 0.5124 | 1.024 |
| wtPwave_timeCentroid_Z_median¹ | n/s | 0.5029 | n/s |
| wtPwave_timeRange_Y_median¹ | n/s | 0.5883 | 1.3780 |
| wtPwave_eccentricity_Y_iqr² | 0.00297 | 0.5352 | n/s |
| wtPwave_timeCentroid_X_median³ | 0.0006 | 0.5640 | 1.0211 |
| wtPwave_timeCentroid_Y_median³ | 0.0002 | 0.6086 | 1.0843 |
| wtQRSwave_circularity_X_iqr | n/s | 0.5056 | n/s |
| wtQRSwave_circularity_Z_iqr | 0.0426 | n/s | n/s |
| wtQRSwave_eccentricity_X_iqr | 0.0011 | 0.5532 | 1.0148 |
| wtQRSwave_eccentricity_X_median | 0.0195 | n/s | n/s |
| wtQRSwave_eccentricity_Y_median | n/s | 0.5032 | n/s |
| wtQRSwave_extent_Z_iqr | n/s | 0.5005 | n/s |
| wtQRSwave_extent_Z_median | 0.0471 | n/s | n/s |
| wtQRSwave_frequencyCentroid_X_iqr | n/s | 0.5233 | n/s |
| wtQRSwave_frequencyCentroid_X_median | 0.0082 | n/s | n/s |
| wtQRSwave_frequencyCentroid_Z_iqr | 0.0031 | 0.5013 | n/s |
| wtQRSwave_frequencyRange_X_median | 0.0061 | n/s | n/s |
| wtQRSwave_frequencyRange_Z_iqr | 0.0246 | n/s | n/s |
| wtQRSwave_orientation_X_iqr | n/s | 0.5079 | n/s |
| wtQRSwave_orientation_Z_median | 0.0222 | 0.5026 | n/s |
| wtQRSwave_timeCentroid_X_iqr | n/s | 0.5121 | n/s |
| wtQRSwave_timeCentroid_Y_iqr | n/s | 0.5075 | n/s |
| wtQRSwave_timeRange_X_iqr | 0.0182 | 0.5227 | n/s |
| wtQRSwave_timeRange_X_median | 0.0331 | 0.5135 | n/s |
| wtQRSwave_timeRange_Z_iqr | 0.0005 | 0.5532 | 1.5787 |
| wtQRSwave_timeCentroid_X_median¹ | 0.0411 | n/s | n/s |
| wtTwave_circularity_X_iqr | n/s | 0.5549 | 1.3547 |
| wtTwave_eccentricity_X_iqr | 0.0122 | 0.5582 | n/s |
| wtTwave_eccentricity_Y_median | 0.0319 | n/s | n/s |
| wtTwave_eccentricity_Z_median | n/s | 0.5013 | n/s |
| wtTwave_extent_X_iqr | 0.0213 | n/s | n/s |
| wtTwave_extent_X_median | 0.0048 | n/s | n/s |
| wtTwave_frequencyCentroid_X_iqr | n/s | 0.5083 | n/s |
| wtTwave_frequencyRange_X_iqr | n/s | 0.5463 | n/s |
| wtTwave_frequencyRange_Y_median | n/s | 0.5005 | n/s |
| wtTwave_orientation_X_iqr | n/s | 0.5163 | n/s |
| wtTwave_orientation_Y_iqr | 0.0394 | n/s | n/s |
| wtTwave_powerCentroid_Z_iqr | 0.0039 | 0.5264 | n/s |
| wtTwave_timeCentroid_X_median | 0.0237 | n/s | n/s |
| wtTwave_timeCentroid_Z_median | 0.0085 | n/s | n/s |
| wtTwave_timeRange_X_iqr | 0.0006 | 0.5436 | 1.0995 |
| wtTwave_circularity_Y_median¹ | 0.0476 | n/s | n/s |
| wtTwave_circularity_Z_median¹ | n/s | 0.5169 | n/s |
| wtTwave_timeCentroid_Y_median¹ | 0.0008 | 0.5460 | 1.0288 |
| wtTwave_timeRange_Y_median¹ | 0.0294 | n/s | n/s |
| wtTwave_timeRange_Z_median¹ | n/s | 0.5088 | n/s |
| wtCohXY_eccentricity | 0.0393 | n/s | n/s |
| wtCohXY_frequencyCentroid | 0.0364 | n/s | n/s |

FA_scenario = LVEDP <= 12 (N = 246) vs >=20 (N = 209)
¹= LVEDP <= 12 (N = 246) vs >=25 (N = 78)
²= LVEDP <= 20 (N = 95) vs CADHealth G1 (N = 122)
³= LVEDP >= 25 (N = 95) vs CADHealth G2 (N = 37)

TABLE 8B

| Feature_name | t-test | AUC | MI |
|---|---|---|---|
| wtPwaveDecay_eccentricity_Decay_X_median | 0.0390 | 0.5046 | n/s |
| wtPwaveDecay_numRegion/s_Decay_X_median | 0.0036 | n/s | n/s |
| wtPwaveDecay_numRegion/sR2_Decay_Y_median | n/s | n/s | n/s |
| wtPwaveDecay_orientation_Decay_X_median | n/s | 0.5099 | n/s |
| wtPwaveDecay_orientation_Decay_Z_median | n/s | n/s | n/s |
| wtPwaveDecay_timeRange_Decay_Y_median | 0.0218 | n/s | n/s |
| wtPwaveDecay_frequencyRange_Decay_Z_median¹ | 0.0450 | n/s | 1.0208 |
| wtPwaveDecay_numRegion/sR2_Decay_Y_median¹ | 0.0426 | n/s | n/s |
| wtPwaveDecay_orientation_Decay_Z_median¹ | 0.0029 | n/s | n/s |
| wtQRSwaveDecay_frequencyCentroid_Decay_X_median | 0.0099 | n/s | n/s |
| wtQRSwaveDecay_orientation_Decay_Z_median | 0.0088 | 0.5112 | n/s |
| wtQRSwaveDecay_timeCentroid_Decay_Z_median | 0.0125 | 0.5141 | 1.0622 |
| wtQRSwaveDecay_circularity_Decay_Y_median¹ | 0.0384 | n/s | n/s |
| wtQRSwaveDecay_extent_Decay_Y_median¹ | 0.0215 | n/s | n/s |
| wtQRSwaveDecay_timeCentroid_Decay_X_median¹ | 0.0393 | n/s | n/s |
| wtQRSwaveDecay_eccentricity_Decay_X_median³ | 0.0033 | 0.5112 | 1.1271 |
| wtTwaveDecay_eccentricity_Decay_Y_median | 0.0020 | 0.5071 | 1.0575 |
| wtTwaveDecay_extent_Decay_X_median | 0.0429 | n/s | n/s |
| wtTwaveDecay_numRegion/sR2_Decay_X_median | n/s | 0.5019 | n/s |
| wtTwaveDecay_powerCentroid_Decay_X_median | 0.0286 | n/s | n/s |
| wtTwaveDecay_timeCentroid_Decay_X_median | 0.0192 | n/s | n/s |
| wtTwaveDecay_timeCentroid_Decay_Y_median | 0.0121 | n/s | n/s |
| wtTwaveDecay_timeCentroid_Decay_Z_median | 0.0046 | 0.5024 | 1.3938 |
| wtTwaveDecay_timeRange_Decay_Y_median | 0.0465 | n/s | n/s |
| wtTwaveDecay_circularity_Decay_X_median¹ | 0.0199 | 0.5130 | n/s |
| wtTwaveDecay_circularity_Decay_Y_median¹ | 0.0387 | n/s | n/s |
| wtTwaveDecay_circularity_Decay_Z_median¹ | 0.0430 | 0.5250 | n/s |
| wtTwaveDecay_eccentricity_Decay_X_median¹ | 0.0327 | n/s | n/s |
| wtTwaveDecay_timeRange_Decay_Z_median¹ | 0.0466 | n/s | 1.0080 |
| wtTwaveDecay_eccentricity_Decay_Z_median² | n/s | 0.6846 | 3.1387 |

FA_scenario = LVEDP <= 12 (N = 246) vs >=20 (N = 209)
* = LVEDP <= 12 (N = 246) vs >=25 (N = 78)
** = LVEDP <= 20 (N = 95) vs CADHealth G1 (N = 122)
*** = LVEDP >= 25 (N = 95) vs CADHealth G2 (N = 37)

TABLE 8C

| Feature_name | t-test | AUC | MI |
|---|---|---|---|
| wtPwaveDist_cdfNormal_std_Z_n/snmedian | 0.0258 | n/s | n/s |
| wtPwaveDist_medianPSD_Z_n/snmedian | n/s | 0.5042 | n/s |
| wtPwaveDist_peakDispPSD_Y_n/snmedian | n/s | 0.5019 | n/s |
| wtPwaveDist_qqsse_Uniform_Y_n/snmedian | n/s | 0.5024 | n/s |
| wtPwaveDist_skewPSD_X_n/snmedian | 0.0393 | n/s | n/s |
| wtPwaveDist_skewPSD_Z_n/snmedian | 0.0299 | n/s | n/s |
| wtPwaveDist_peakWidthPSD_Y_n/snmedian¹ | 0.0046 | 0.5530 | n/s |
| wtQRSwaveDist_cdfNormal_L1norm_Y_n/snmedian | 0.0396 | n/s | n/s |
| wtQRSwaveDist_cdfNormal_std_Y_n/snmedian | 0.0283 | n/s | n/s |
| wtQRSwaveDist_entropy_X_n/snmedian | 0.0198 | 0.5050 | n/s |
| wtQRSwaveDist_pdfKernel_L1norm_X_n/snmedian | 0.0142 | 0.5238 | 1.1408 |
| wtQRSwaveDist_pdftKernel_std_X_n/snmedian | n/s | 0.5091 | n/s |
| wtQRSwaveDist_peakDispPSD_X_n/snmedian | n/s | 0.5040 | n/s |
| wtQRSwaveDist_peakWidthPSD_X_n/snmedian | 0.0069 | n/s | n/s |
| wtQRSwaveDist_qqr2_Normal_Y_n/snmedian | 0.0375 | n/s | n/s |
| wtQRSwaveDist_qqr2_Uniform_Y_n/snmedian | 0.0324 | n/s | n/s |
| wtQRSwaveDist_qqslop_Uniform_X_n/snmedian | n/s | 0.5190 | n/s |
| wtQRSwaveDist_qqsse_Normal_X_n/snmedian | n/s | 0.5273 | n/s |
| wtQRSwaveDist_qqsse_Uniform_Y_n/snmedian | 0.0215 | n/s | n/s |
| wtQRSwaveDist_stdPSD_X_n/snmedian | n/s | 0.5246 | n/s |
| wtQRSwaveDist_qqslop_Normal_X_n/snmedian* | n/s | 0.5145 | 1.1027 |
| wtTwaveDist_pdftKernel_std_X_n/snmedian | n/s | 0.5005 | n/s |
| wtTwaveDist_peakWidthPSD_Y_n/snmedian | n/s | 0.5005 | n/s |

FA_scenario = LVEDP <= 12 (N = 246) vs >=20 (N = 209)
*= LVEDP <= 12 (N = 246) vs >=25 (N = 78)

Tables 9A, 9B, and 9C each shows a list of power spectral-based features having been determined to have utility in estimating the presence and non-presence of significant CAD in an algorithm executing in a clinical evaluation system. The features of Tables 9A, 9B, and 9C and corresponding classifiers have been validated to have clinical performance comparable to the gold standard invasive method to measure CAD.

TABLE 9A

| Feature_name | t-test | AUC | MI |
|---|---|---|---|
| wtPwave_eccentricity_Z_median | 0.0495 | 0.5031 | n/s |
| wtPwave_frequencyCentroid_Z_median | n/s | n/s | 1.048 |
| wtPwave_surfaceArea_Z_iqr | n/s | n/s | 1.0636 |
| wtPwaveDist_peakDispPSD_Z_median | 0.005 | n/s | n/s |
| wtQRSwave_circularity_Z_median | 0.0316 | 0.5172 | n/s |
| wtQRSwave_eccentricity_Z_median | n/s | 0.501 | n/s |
| wtQRSwave_extent_X_median | n/s | 0.5174 | n/s |
| wtQRSwave_extent_Z_median | 0.0412 | n/s | n/s |
| wtQRSwave_frequencyCentroid_Y_iqr | 0.0413 | n/s | n/s |
| wtQRSwave_orientation_Z_median | n/s | 0.5164 | n/s |
| wtQRSwave_timeRange_X_median | n/s | n/s | 1.2334 |
| wtTwave_circularity_X_iqr | n/s | n/s | 1.0514 |
| wtTwave_circularity_Y_median | 0.041 | 0.5071 | n/s |
| wtTwave_circularity_Z_median | 0.0263 | 0.5007 | n/s |
| wtTwave_eccentricity_Y_iqr | 0.0229 | n/s | n/s |
| wtTwave_eccentricity_Y_median | 0.0342 | n/s | n/s |
| wtTwave_eccentricity_Z_median | 0.026 | n/s | n/s |
| wtTwave_extent_Z_iqr | n/s | n/s | 1.2081 |
| wtTwave_frequencyRange_Z_median | 0.0127 | 0.5105 | n/s |
| wtTwave_timeCentroid_Z_median | n/s | n/s | 1.0108 |
| wtTwave_timeRange_Y_median | 0.0216 | n/s | n/s |
| wtTwave_timeRange_Z_median | 0.0007 | 0.5351 | 1.0061 |
| wt_ppg_circularity_iqr | n/s | n/s | 1.160269 |
| wt_ppg_eccentricity_iqr | 0.004926 | 0.52172 | 1.157167 |
| wt_ppg_eccentricity_median | 0.038521 | n/s | n/s |
| wt_ppg_extent_median | 0.002601 | 0.556672 | n/s |
| wt_ppg_frequencyCentroid_iqr | 0.036022 | 0.533689 | 1.162764 |
| wt_ppg_frequencyRange_Decay_median | 0.015936 | 0.537363 | 1.359679 |
| wt_ppg_frequencyRange_iqr | 0.02965 | n/s | n/s |
| wt_ppg_frequencyRange_median | 0.013908 | n/s | 1.142739 |
| wt_ppg_numRegions_Decay_median | 0.014822 | 0.535842 | 1.448836 |
| wt_ppg_numRegionsR2_Decay_median | 9.19E−05 | 0.552833 | 1.319685 |
| wt_ppg_orientation_iqr | 0.038469 | 0.514786 | 1.133562 |
| wt_ppg_orientation_median | 0.009537 | 0.532602 | 1.245184 |
| wt_ppg_powerCentroid_Decay_median | 0.005164 | n/s | 1.497595 |
| wt_ppg_powerCentroid_iqr | 0.012148 | n/s | n/s |
| wt_ppg_powerCentroid_median | 0.007896 | n/s | 1.427262 |
| wt_ppg_timeCentroid_iqr | 0.030764 | 0.500493 | n/s |
| wt_ppg_timeCentroid_median | 0.036219 | 0.511304 | n/s |
| wt_ppg_timeRange_iqr | n/s | 0.518563 | n/s |
| wtCohcircularity_XY | 0.0304 | n/s | n/s |
| wtCohextent_XY | 0.015 | n/s | 2.153 |
| wtCohXY_frequencyCentroid | n/s | n/s | 1.1219 |
| wtCohXZ_frequencyRange | 0.0274 | n/s | 1.1339 |
| wtCohYZ_frequencyRange | 0.0349 | n/s | n/s |
| wt_vpg_circularity_iqr | n/s | n/s | 1.0903 |
| wt_vpg_Dist_meanPSD_median | 0.0402 | n/s | n/s |
| wt_vpg_Dist_medianPSD_median | 0.0336 | n/s | n/s |
| wt_vpg_Dist_pdftKernel_std_median | 0.0410 | n/s | 1.1481 |
| wt_vpg_Dist_peakDispPSD_median | n/s | n/s | 1.0378 |
| wt_vpg_Dist_peakWidthPSD_median | n/s | n/s | 1.3016 |
| wt_vpg_Dist_skewPSD_median | 0.0115 | 0.5101 | n/s |
| wt_vpg_Dist_sumPSD_median | 0.0134 | n/s | n/s |
| wt_vpg_eccentricity_Decay_median | 0.0002 | 0.5435 | 1.4145 |
| wt_vpg_eccentricity_median | n/s | 0.5085 | n/s |
| wt_vpg_extent_iqr | 0.0496 | n/s | n/s |
| wt_vpg_extent_median | 0.0381 | n/s | n/s |
| wt_vpg_frequencyCentroid_median | n/s | n/s | 1.0421 |
| wt_vpg_frequencyRange_median | n/s | 0.5000 | n/s |
| wt_vpg_numRegions_Decay_median | n/s | n/s | 1.3255 |
| wt_vpg_numRegionsR2_Decay_median | n/s | n/s | 1.0586 |
| wt_vpg_orientation_Decay_median | 0.0195 | n/s | 1.5050 |
| wt_vpg_orientation_median | n/s | n/s | 1.0223 |
| wt_vpg_timeCentroid_Decay_median | 0.0013 | n/s | n/s |

FA scenario = significant CAD (e.g., defined as >70% blockage and/or FFR <0.8) (N = 464; 232 CAD positives and 232 CAD negatives (½ single and ½ multi-vessel disease) (½ are males and ½ are females)

TABLE 9B

| Feature_name | t-test | AUC | MI |
|---|---|---|---|
| wtQRSwaveDecay_circularity_Decay_Z_median | 0.0148 | n/s | n/s |
| wtQRSwaveDecay_eccentricity_Decay_Z_median | 0.0195 | 0.5118 | 1.3441 |
| wtQRSwaveDecay_orientation_Decay_Z_median | n/s | 0.5013 | n/s |
| wtQRSwaveDecay_surfaceArea_Decay_Z_median | n/s | 0.5107 | n/s |
| wtQRSwaveDecay_timeCentroid_Decay_Z_median | 0.0493 | n/s | n/s |
| wtTwaveDecay_circularity_Decay_Y_median | 0.0403 | n/s | n/s |
| wtTwaveDecay_circularity_Decay_Z_median | 0.0465 | 0.5171 | n/s |
| wtTwaveDecay_eccentricity_Decay_X_median | n/s | n/s | 1.2273 |
| wtTwaveDecay_eccentricity_Decay_Y_median | 0.0162 | n/s | n/s |
| wtTwaveDecay_eccentricity_Decay_Z_median | 0.0024 | n/s | n/s |
| wtTwaveDecay_extent_Decay_X_median | n/s | n/s | 1.4631 |
| wtTwaveDecay_timeRange_Decay_Z_median | 0.0011 | 0.5254 | n/s |

FA scenario = significant CAD (e.g., defined as >70% blockage and/or FFR <0.8) (N = 464; 232 CAD positives and 232 CAD negatives (½ single and ½ multi-vessel disease) (½ are males and ½ are females)

TABLE 9C

| Feature_name | t-test | AUC | MI |
|---|---|---|---|
| wtQRSwaveDist_cdfNormal_L1norm_X_median | 0.0124 | 0.5053 | n/s |
| wtQRSwaveDist_cdfNormal_L1norm_Z_median | 0.026 | 0.5093 | n/s |
| wtQRSwaveDist_cdfNormal_std_X_median | 0.0162 | 0.5076 | n/s |
| wtQRSwaveDist_cdfNormal_std_Z_median | 0.0329 | 0.5039 | n/s |
| wtQRSwaveDist_entropy_Z_median | n/s | n/s | 1.2943 |
| wtQRSwaveDist_kurtPSD_X_median | n/s | n/s | 1.3279 |
| wtQRSwaveDist_kurtPSD_Z_median | n/s | n/s | 1.0383 |
| wtQRSwaveDist_qqr2_Normal_X_median | 0.0173 | 0.5131 | n/s |
| wtQRSwaveDist_qqr2_Normal_Z_median | 0.0311 | n/s | n/s |
| wtQRSwaveDist_qqr2_Uniform_X_median | 0.0236 | n/s | n/s |
| wtQRSwaveDist_qqr2_Uniform_Z_median | 0.0344 | n/s | n/s |
| wtQRSwaveDist_qqsse_Uniform_X_median | n/s | 0.5016 | n/s |
| wtTwaveDist_entropy_Z_median | 0.0212 | 0.5005 | n/s |
| wtTwaveDist_kurtPSD_Y_median | n/s | n/s | 1.2395 |
| wtTwaveDist_medianPSD_Y_median | n/s | n/s | 1.0892 |
| wtTwaveDist_pdftKernel_std_Z_median | 0.027 | 0.518 | n/s |
| wtTwaveDist_peakWidthPSD_Z_median | 0.0196 | 0.5105 | n/s |
| wtTwaveDist_qqslop_Normal_Z_median | 0.0296 | 0.5155 | n/s |
| wtTwaveDist_qqslop_Uniform_Z_median | 0.0296 | 0.5159 | n/s |
| wtTwaveDist_qqsse_Normal_Z_median | 0.0396 | n/s | n/s |
| wtTwaveDist_stdPSD_Z_median | 0.0258 | 0.5103 | n/s |
| wt_ppg_Dist_entropy | 0.0118 | n/s | n/s |
| wt_ppg_Dist_fpeakRatioPSD | 0.0029 | 0.5057 | 1.0307 |
| wt_ppg_Dist_kurtPSD | 0.0001 | n/s | 1.3244 |
| wt_ppg_Dist_meanPSD | 0.0423 | n/s | n/s |
| wt_ppg_Dist_numpeakPSD | 0.0070 | n/s | 1.4628 |
| wt_ppg_Dist_pdfKernel_L1norm | 0.0056 | 0.5371 | 1.1040 |
| wt_ppg_Dist_pdftKernel_dpowerPeak | 0.0014 | 0.5432 | 1.3374 |
| wt_ppg_Dist_pdftKernel_numpeak | 0.0241 | n/s | 1.0871 |
| wt_ppg_Dist_pdftKernel_std | 0.0058 | 0.5294 | 1.1637 |
| wt_ppg_Dist_peakDispPSD | 0.0431 | n/s | n/s |
| wt_ppg_Dist_peakDispRate | 0.0025 | 0.5357 | n/s |
| wt_ppg_Dist_peakRatioPSD | 0.0014 | 0.5440 | n/s |
| wt_ppg_Dist_peakWidthPSD | 0.0018 | 0.5506 | 1.4030 |
| wt_ppg_Dist_qqr2_Uniform | 0.0109 | n/s | n/s |
| wt_ppg_Dist_qqslop_Normal | 0.0168 | 0.5284 | 1.1581 |
| wt_ppg_Dist_qqslop_Uniform | 0.0146 | 0.5355 | 1.0297 |
| wt_ppg_Dist_qqsse_Normal | n/s | 0.5063 | n/s |
| wt_ppg_Dist_skewPSD | 0.0367 | n/s | n/s |
| wt_ppg_Dist_stdPSD | 0.0171 | 0.5293 | 1.5332 |

FA scenario = significant CAD (e.g., defined as >70% blockage and/or FFR <0.8) (N = 464; 232 CAD positives and 232 CAD negatives (½ single and ½ multi-vessel disease) (½ are males and ½ are females)

The determination that certain wavelet-based features have clinical utility in estimating the presence and non-presence of elevated LVEDP or the presence and non-presence of significant CAD provides a basis for the use of these wavelet-based features or parameters, as well as other features described herein, in estimating for the presence or non-presence and/or severity and/or localization of other diseases, medical conditions, or indications of either particularly, though not limited to, heart disease or conditions described herein.

The experimental results further indicate that intermediary data or parameters of wavelet-based features also have clinical utility in diagnostics as well as treatment, controls, monitoring, and tracking applications.

Example Clinical Evaluation System

FIG. 15A shows an example clinical evaluation system 1500 (also referred to as a clinical and diagnostic system) that implements the modules of FIG. 1 to non-invasively compute wavelet-based features or parameters, along with other features or parameters, to generate, via a classifier (e.g., machine-learned classifier), one or more metrics associated with the physiological state of a patient or subject according to an embodiment. Indeed, the feature modules (e.g., of FIGS. 1, 4-14) can be generally viewed as a part of a system (e.g., the clinical evaluation system 1500) in which any number and/or types of features may be utilized for a disease state, medical condition, an indication of either, or combination thereof that is of interest, e.g., with different embodiments having different configurations of feature modules. This is additionally illustrated in FIG. 15A, where the clinical evaluation system 1500 is of a modular design in which disease-specific add-on modules 1502 (e.g., to assess for elevated LVEDP or mPAP, CAD, PH/PAH, abnormal LVEF, HFpEF, and others described herein) are capable of being integrated alone or in multiple instances with a singular platform (i.e., a base system 1504) to realize system 1500's full operation. The modularity allows the clinical evaluation system 1500 to be designed to leverage the same synchronously acquired biophysical signals and data set and base platform to assess for the presence of several different diseases as such disease-specific algorithms are developed, thereby reducing testing and certification time and cost.

In various embodiments, different versions of the clinical evaluation system 1500 may implement the assessment system 103 (FIG. 1) by having included containing different feature computation modules that can be configured for a given disease state(s), medical condition(s), or indicating condition(s) of interest. In another embodiment, the clinical evaluation system 1500 may include more than one assessment system 103 and maybe selectively utilized to generate different scores specific to a classifier 116 of that engine 103. In this way, the modules of FIGS. 1 and 15 in a more general sense may be viewed as one configuration of a modular system in which different and/or multiple engines 103, with different and/or multiple corresponding classifiers 116, may be used depending on the configuration of module desired. As such, any number of embodiments of the modules of FIG. 1, with or without wavelet-based specific feature(s), may exist.

In FIG. 15A, System 1500 can analyze one or more biophysical-signal data sets (e.g., 110) using machine-learned disease-specific algorithms to assess for the likelihood of elevated LVEDP, as one example, of pathology or abnormal state. System 1500 includes hardware and software components that are designed to work together in combination to facilitate the analysis and presentation of an estimation score using the algorithm to allow a physician to use that score, e.g., to assess for the presence or non-presence of a disease state, medical condition, or an indication of either.

The base system 1504 can provide a foundation of functions and instructions upon which each add-on module 1502 (which includes the disease-specific algorithm) then interface to assess for the pathology or indicating condition. The base system 1504, as shown in the example of FIG. 15A, includes a base analytical engine or analyzer 1506, a web-service data transfer API 1508 (shown as “DTAPI” 1508), a report database 1510, a web portal service module 1513, and the data repository 111 (shown as 112a).

Data repository 112a, which can be cloud-based, stores data from the signal capture system 102 (shown as 102b). Biophysical signal capture system 102b, in some embodiments, is a reusable device designed as a single unit with a seven-channel lead set and photoplethysmogram (PPG) sensor securely attached (i.e., not removable). Signal capture system 102b, together with its hardware, firmware, and software, provides a user interface to collect patient-specific metadata entered therein (e.g., name, gender, date of birth, medical record number, height, and weight, etc.) to synchronously acquire the patient's electrical and hemodynamic signals. The signal capture system 102b may securely transmit the metadata and signal data as a single data package directly to the cloud-based data repository. The data repository 112a, in some embodiments, is a secure cloud-based database configured to accept and store the patient-specific data package and allow for its retrieval by the analytical engines or analyzer 1506 or 1514.

Base analytical engine or analyzer 1506 is a secure cloud-based processing tool that may perform quality assessments of the acquired signals (performed via “SQA” module 1516), the results of which can be communicated to the user at the point of care. The base analytical engine or analyzer 1506 may also perform pre-processing (shown via pre-processing module 1518) of the acquired biophysical signals (e.g., 110—see FIG. 1). Web portal 1513 is a secure web-based portal designed to provide healthcare providers access to their patient's reports. An example output of the web portal 1513 is shown by visualization 1536. The report databases (RD) 1512 is a secure database and may securely interface and communicate with other systems, such as a hospital or physician-hosted, remotely hosted, or remote electronic health records systems (e.g., Epic, Cerner, Allscrips, CureMD, Kareo, etc.) so that output score(s) (e.g., 118) and related information may be integrated into and saved with the patient's general health record. In some embodiments, web portal 1513 is accessed by a call center to provide the output clinical information over a telephone. Database 1512 may be accessed by other systems that can generate a report to be delivered via the mail, courier service, personal delivery, etc.

Add-on module 1502 includes a second part 1514 (also referred to herein as the analytical engine (AE) or analyzer 1514 and shown as “AE add-on module” 1514) that operates with the base analytical engine (AE) or analyzer 1506. Analytical engine (AE) or analyzer 1514 can include the main function loop of a given disease-specific algorithm, e.g., the feature computation module 1520, the classifier model 1524 (shown as “Ensemble” module 1524), and the outlier assessment and rejection module 1524 (shown as “Outlier Detection” module 1524). In certain modular configurations, the analytical engines or analyzers (e.g., 1506 and 1514) may be implemented in a single analytical engine module.

The main function loop can include instructions to (i) validate the executing environment to ensure all required environment variables values are present and (ii) execute an analysis pipeline that analyzes a new signal capture data file comprising the acquired biophysical signals to calculate the patient's score using the disease-specific algorithm. To execute the analysis pipeline, AE add-on module 1514 can include and execute instructions for the various feature modules 114 and classifier module 116 as described in relation to FIG. 1 to determine an output score (e.g., 118) of the metrics associated with the physiological state of a patient. The analysis pipeline in the AE add-on module 1514 can compute the features or parameters (shown as “Feature Computation” 1520) and identifies whether the computed features are outliers (shown as “Outlier Detection” 1522) by providing an outlier detection return for a signal-level response of outlier vs non-outlier based on the feature. The outliers may be assessed with respect to the training data set used to establish the classifier (of module 116). AE add-on module 1514 may generate the patient's output score (e.g., 118) (e.g., via classifier module 1524) using the computed values of the features and classifier models. In the example of an evaluation algorithm for the estimation of elevated LVEDP, the output score (e.g., 118) is an LVEDP score. For the estimation of CAD, the output score (e.g., 118) is a CAD score.

The clinical evaluation system 1500 can manage the data within and across components using the web-service DTAPIs 1508 (also may be referred to as HCPP web services in some embodiments). DTAPIs 1508 may be used to retrieve acquired biophysical data sets from, and to store signal quality analysis results to, the data repository 112a. DTAPIs 1508 may also be invoked to retrieve and provide the stored biophysical data files to the analytical engines or analyzers (e.g., 1506, 1514), and the results of the analytical engine's analysis of the patient signals may be transferred using DTAPI 1508 to the report database 1510. DTAPIs 1508 may also be used, upon a request by a healthcare professional, to retrieve a given patient data set to the web portal module 1513, which may present a report to the healthcare practitioner for review and interpretation in a secure web-accessible interface.

Clinical evaluation system 1500 includes one or more feature libraries 1526 that store the wavelet-based features 120 and various other features of the feature modules 122. The feature libraries 1526 may be a part of the add-on modules 1502 (as shown in FIG. 15A) or the base system 1504 (not shown) and are accessed, in some embodiments, by the AE add-on module 1514.

Further details of the modularity of modules and various configurations are provided in U.S. Provisional Patent Application No. 63/235,960, filed Aug. 19, 2021, entitled “Modular Disease Assessment System,” which is hereby incorporated by reference herein in its entirety.

Example Operation of the Modular Clinical Evaluation System

FIG. 15B shows a schematic diagram of the operation and workflow of the analytical engines or analyzers (e.g., 1506 and 1514) of the clinical evaluation system 1500 of FIG. 15A in accordance with an illustrative embodiment.

Signal quality assessment/rejection (1530). Referring to FIG. 15B, the base analytical engine or analyzer 1506 assesses (1530), via SQA module 1516, the quality of the acquired biophysical-signal data set while the analysis pipeline is executing. The results of the assessment (e.g., pass/fail) are immediately returned to the signal capture system's user interface for reading by the user. Acquired signal data that meet the signal quality requirements are deemed acceptable (i.e., “pass”) and further processed and subjected to analysis for the presence of metrics associated with the pathology or indicating condition (e.g., elevated LVEDP or mPAP, CAD, PH/PAH, abnormal LVEF, HFpEF) by the AE add-on module 1514. Acquired signals deemed unacceptable are rejected (e.g., “fail”), and a notification is immediately sent to the user to inform the user to immediately obtain additional signals from the patient (see FIG. 2).

The base analytical engine or analyzer 1506 performs two sets of assessments for signal quality, one for the electrical signals and one for the hemodynamic signals. The electrical signal assessment (1530) confirms that the electrical signals are of sufficient length, that there is a lack of high-frequency noise (e.g., above 170 Hz), and that there is no power line noise from the environment. The hemodynamic signal assessment (1530) confirms that the percentage of outliers in the hemodynamic data set is below a pre-defined threshold and that the percentage and maximum duration that the signals of the hemodynamic data set are railed or saturated is below a pre-defined threshold.

Feature Value Computation (1532). The AE add-on module 1514 performs feature extraction and computation to calculate feature output values. In the example of the LVEDP algorithm, the AE add-on module 1514 determines, in some embodiments, a total of 446 feature outputs belonging to 18 different feature families (e.g., generated in modules 120 and 122), including the wavelet-based features (e.g., generated in module 120). For the CAD algorithm, an example implementation of the AE add-on module 1214 determines a set of features, including 456 features corresponding to the same 18 feature families.

Additional descriptions of the various features, including those used in the LVEDP algorithm and other features and their feature families, are described in U.S. Provisional Patent Application No. 63/235,960, filed Aug. 23, 2021, entitled “Method and System to Non-Invasively Assess Elevated Left Ventricular End-Diastolic Pressure”; U.S. Provisional Patent Application No. 63/236,072, filed Aug. 23, 2021, entitled “Methods and Systems for Engineering Visual Features From Biophysical Signals for Use in Characterizing Physiological Systems”; U.S. Provisional Patent Application No. 63/235,963, filed Aug. 23, 2021, entitled “Methods and Systems for Engineering Power Spectral Features From Biophysical Signals for Use in Characterizing Physiological Systems”; U.S. Provisional Patent Application No. 63/235,966, filed Aug. 23, 2021, entitled “Method and System for Engineering Rate-Related Features From Biophysical Signals for Use in Characterizing Physiological Systems”; a U.S. Provisional Patent Application No. 63/130,324, titled “Method and System to Assess Disease Using Cycle Variability Analysis of Cardiac and Photoplethysmographic Signals”; U.S. Provisional Patent Application no. 63/235,971, filed Aug. 23, 2021, entitled “Methods and Systems for Engineering photoplethysmographic Waveform Features for Use in Characterizing Physiological Systems”; U.S. Provisional Patent Application No. 63/236,193, filed Aug. 23, 2021, entitled “Methods and Systems for Engineering Cardiac Waveform Features From Biophysical Signals for Use in Characterizing Physiological Systems”; U.S. Provisional Patent Application No. 63/235,974, filed Aug. 23, 2021, entitled “Methods and Systems for Engineering Conduction Deviation Features From Biophysical Signals for Use in Characterizing Physiological Systems”, each of which is hereby incorporated by reference herein in its entirety.

Classifier Output Computation (1534). The AE add-on module 1514 then uses the calculated feature outputs in classifier models (e.g., machine-learned classifier models) to generate a set of model scores. The AE add-on module 1514 joins the set of model scores in an ensemble of the constituent models, which, in some embodiments, averages the output of the classifier models as shown in Equation 5 in the example of the LVEDP algorithm.

$$Ensemble estimation = \frac{{Model}_{1} + {Model}_{2} + … + {Model}_{n}}{n} \qquad ( Equation 5 )$$

In some embodiments, classifier models may include models that are developed based on ML techniques described in U.S. Patent Publication No. 20190026430, entitled “Discovering Novel Features to Use in Machine Learning Techniques, such as Machine Learning Techniques for Diagnosing Medical Conditions”; or U.S. Patent Publication No. 20190026431, entitled “Discovering Genomes to Use in Machine Learning Techniques,” each of which is hereby incorporated by reference herein in its entirety.

In the example of the LVEDP algorithm, thirteen (13) machine-learned classifier models are each calculated using the calculated feature outputs. The 13 classifier models include four ElasticNet machine-learned classifier models, four RandomForestClassifier machine-learned classifier models, and five extreme gradient boosting (XGB) classifier models. In some embodiments, the patient's metadata information, such as age, gender, BMI value, may be used. The output of the ensemble estimation may be a continuous score. The score may be shifted to a threshold value of zero by subtracting the threshold value for presentation within the web portal. The threshold value may be selected as a trade-off between sensitivity and specificity. The threshold may be defined within the algorithm and used as the determination point for test positive (e.g., “Likely Elevated LVEDP”) and test negative (e.g., “Not Likely Elevated LVEDP”) condition.

In some embodiments, the analytical engine or analyzer can fuse the set of model scores with a body mass index-based adjustment or an adjustment based on age or gender. For example, the analytical engine or analyzer can average the model estimation with a sigmoid function of the patient BMI having the form sigmoid(x)=1/1+e^{-x}.

Physician Portal Visualization (1536). The patient's report may include a visualization 1536 of the acquired patient data and signals and the results of the disease analyses. The analyses are presented, in some embodiments, in multiple views in the report. In the example shown in FIG. 15B, the visualization 1536 includes a score summary section 1540 (shown as “Patient LVEDP Score Summary” section 1540), a threshold section 1542 (shown as “LVEDP Threshold Statistics” section 1542), and a frequency distribution section 1544 (shown as “Frequency Distribution” section 1508). A healthcare provider, e.g., a physician, can review the report and interpret it to provide a diagnosis of the disease or to generate a treatment plan.

The healthcare portal may list a report for a patient if a given patient's acquired signal data set meets the signal quality standard. The report may indicate a disease-specific result (e.g., elevated LVEDP) being available if the signal analysis could be performed. The patient's estimated score (shown via visual element 118a, 118b, 118c) for the disease-specific analysis may be interpreted relative to an established threshold.

In the score summary section 1540 shown in the example of FIG. 15B, the patient's score 118a and associated threshold are superimposed on a two-tone color bar (e.g., shown in section 1540) with the threshold located at the center of the bar with a defined value of “0” representing the delineation between test positive and test negative. The left of the threshold may be lightly shaded light and indicates a negative test result (e.g., “Not Likely Elevated LVEDP”), while to the right of the threshold may be darkly shaded to indicate a positive test result (e.g., “Likely Elevated LVEDP”).

The threshold section 1542 shows reported statistics of the threshold as provided to a validation population that defines the sensitivity and specificity for the estimation of the patient score (e.g., 118). The threshold is the same for every test regardless of the individual patient's score (e.g., 118), meaning that every score, positive or negative, may be interpreted for accuracy in view of the provided sensitivity and specificity information. The score may change for a given disease-specific analysis as well with the updating of the clinical evaluation.

The frequency distribution section 1544 illustrates the distribution of all patients in two validation populations (e.g., (i) a non-elevated population to indicate the likelihood of a false positive estimation and (ii) an elevated population to indicate a likelihood of a false negative estimation). The graphs (1546, 1548) are presented as smooth histograms to provide context for interpreting the patient's score 118 (e.g., 118b, 118c) relative to the test performance validation population patients.

The frequency distribution section 1540 includes a first graph 1546 (shown as “Non-Elevated LVEDP Population” 1546) that shows the score (118b), indicating the likelihood of the non-presence of the disease, condition, or indication, within a distribution of a validation population having non-presence of that disease, condition, or indication and a second graph 1548 (shown as “Elevated LVEDP Population” 1548) that shows the score (118c), indicates the likelihood of the presence of the disease, condition, or indication, within a distribution of validation population having the presence of that disease, condition, or indication. In the example of the assessment of elevated LVDEP, the first graph 1546 shows a non-elevated LVEDP distribution of the validation population that identifies the true negative (TN) and false positive (FP) areas. The second graph 1548 shows an elevated LVEDP distribution of the validation population that identifies the false negative (TN) and true positive (FP) areas.

The frequency distribution section 1540 also includes interpretative text of the patient's score relative to other patients in a validation population group (as a percentage). In this example, the patient has an LVEDP score of −0.08, which is located to the left side of the LVEDP threshold, indicating that the patient has “Not Likely Elevated LVEDP.”

The report may be presented in the healthcare portal, e.g., to be used by a physician or healthcare provider in their diagnosis for indications of left-heart failure. The indications include, in some embodiments, a probability or a severity score for the presence of a disease, medical condition, or an indication of either.

Outlier Assessment and Rejection Detection (1538). Following the AE add-on module 1514 computing the feature value outputs (in process 1532) and prior to their application to the classifier models (in process 1534), the AE add-on module 1514 is configured in some embodiments to perform outlier analysis (shown in process 1538) of the feature value outputs. Outlier analysis evaluation process 1538 executes a machine-learned outlier detection module (ODM), in some embodiments, to identify and exclude anomalous acquired biophysical signals by identifying and excluding anomalous feature output values in reference to the feature values generated from the validation and training data. The outlier detection module assesses for outliers that present themselves within sparse clusters at isolated regions that are out of distribution from the rest of the observations. Process 1538 can reduce the risk that outlier signals are inappropriately applied to the classifier models and produce inaccurate evaluations to be viewed by the patient or healthcare provider. The accuracy of the outlier module has been verified using hold-out validation sets in which the ODM is able to identify all the labeled outliers in a test set with the acceptable outlier detection rate (ODR) generalization.

While the methods and systems have been described in connection with certain embodiments and specific examples, it is not intended that the scope be limited to the particular embodiments set forth, as the embodiments herein are intended in all respects to be illustrative rather than restrictive. The wavelet-based features discussed herein may ultimately be employed to make, or to assist a physician or other healthcare provider in making, noninvasive diagnoses or determinations of the presence or non-presence and/or severity of other diseases, medical conditions, or indication of either, such as, e.g., coronary artery disease, pulmonary hypertension and other pathologies as described herein using similar or other development approaches. In addition, the example analysis, including the wavelet-based features, can be used in the diagnosis and treatment of other cardiac-related pathologies and indicating conditions as well as neurological-related pathologies and indicating conditions, such assessment can be applied to the diagnosis and treatment (including surgical, minimally invasive, and/or pharmacologic treatment) of any pathologies or indicating conditions in which a biophysical signal is involved in any relevant system of a living body. One example in the cardiac context is the diagnosis of CAD, and other diseases, medical condition, or indicating conditions disclosed herein and its treatment by any number of therapies, alone or in combination, such as the placement of a stent in a coronary artery, the performance of an atherectomy, angioplasty, prescription of drug therapy, and/or the prescription of exercise, nutritional and other lifestyle changes, etc. Other cardiac-related pathologies or indicating conditions that may be diagnosed include, e.g., arrhythmia, congestive heart failure, valve failure, pulmonary hypertension (e.g., pulmonary arterial hypertension, pulmonary hypertension due to left heart disease, pulmonary hypertension due to lung disease, pulmonary hypertension due to chronic blood clots, and pulmonary hypertension due to other diseases such as blood or other disorders), as well as other cardiac-related pathologies, indicating conditions and/or diseases. Non-limiting examples of neurological-related diseases, pathologies or indicating conditions that may be diagnosed include, e.g., epilepsy, schizophrenia, Parkinson's Disease, Alzheimer's Disease (and all other forms of dementia), autism spectrum (including Asperger syndrome), attention deficit hyperactivity disorder, Huntington's Disease, muscular dystrophy, depression, bipolar disorder, brain/spinal cord tumors (malignant and benign), movement disorders, cognitive impairment, speech impairment, various psychoses, brain/spinal cord/nerve injury, chronic traumatic encephalopathy, cluster headaches, migraine headaches, neuropathy (in its various forms, including peripheral neuropathy), phantom limb/pain, chronic fatigue syndrome, acute and/or chronic pain (including back pain, failed back surgery syndrome, etc.), dyskinesia, anxiety disorders, indicating conditions caused by infections or foreign agents (e.g., Lyme disease, encephalitis, rabies), narcolepsy and other sleep disorders, post-traumatic stress disorder, neurological conditions/effects related to stroke, aneurysms, hemorrhagic injury, etc., tinnitus and other hearing-related diseases/indicating conditions and vision-related diseases/indicating conditions.

In addition, the clinical evaluation system described herein may be configured to analyze biophysical signals such as an electrocardiogram (ECG), electroencephalogram (EEG), gamma synchrony, respiratory function signals, pulse oximetry signals, perfusion data signals; quasi-periodic biological signals, fetal ECG signals, blood pressure signals; cardiac magnetic field signals, heart rate signals, among others.

Further examples of processing that may be used with the exemplified method and system disclosed herein are described in: U.S. Pat. Nos. 9,289,150; 9,655,536; 9,968,275; 8,923,958; 9,408,543; 9,955,883; 9,737,229; 10,039,468; 9,597,021; 9,968,265; 9,910,964; 10,672,518; 10,566,091; 10,566,092; 10,542,897; 10,362,950; 10,292,596; 10,806,349; U.S. Patent Publication nos. 2020/0335217; 2020/0229724; 2019/0214137; 2018/0249960; 2019/0200893; 2019/0384757; 2020/0211713; 2019/0365265; 2020/0205739; 2020/0205745; 2019/0026430; 2019/0026431; PCT Publication nos. WO2017/033164; WO2017/221221; WO2019/130272; WO2018/158749; WO2019/077414; WO2019/130273; WO2019/244043; WO2020/136569; WO2019/234587; WO2020/136570; WO2020/136571; U.S. patent application Ser. Nos. 16/831,264; 16/831,380; 17/132,869; PCT Application nos. PCT/IB2020/052889; PCT/IB2020/052890, each of which is hereby incorporated by reference herein in its entirety.

What is claimed is:

1. A method for non-invasively estimating values of one or more metrics associated with a disease state or abnormal condition, the method comprising:
acquiring, by one or more processors, a biophysical-signal data set of a subject comprising one or more first biophysical signals;
determining, by the one or more processors, values of wavelet-based features or parameters that characterize properties or geometric shapes of a binarized data object generated from a wavelet transform of the biophysical-signal data set;
determining, by the one or more processors, an estimated value for a presence of the disease state or abnormal condition-based, in part, on the determined values of the one or more wavelet associated properties, wherein the estimated value for the of the disease state or abnormal condition is used in a model to non-invasively estimate the presence of an expected disease state or condition,
wherein the estimated value is subsequently outputted for use in a diagnosis of the expected disease state or condition or to direct treatment of the expected disease state or condition.

2. A method of claim 1, wherein determining the values of the wavelet-based features or parameters comprises:
determining, by the one or more processors, a wavelet-based model of a plurality of identified periodic cycles of a signal of the biophysical signal data set;
generating, by the one or more processors, a spectral image or data of the wavelet-based model; and
determining, by the one or more processors, one or more values of features extracted from two or three-dimensional objects identified within the spectral image or data.

3. The method of claim 2, wherein the spectral image or data is converted to a binarized image or data by a threshold operator, and wherein the one or more values of the features are extracted from two or three-dimensional objects identified in one or more binarized regions of the threshold spectral image or data.

4. The method of claim 2, wherein the spectral image or data is converted to a plurality of binarized image or data by a plurality of corresponding threshold operators, and wherein the one or more values of the features are extracted from two or three-dimensional objects identified in one or more binarized regions of the plurality of threshold spectral image or data.

5. The method of claim 2, wherein the spectral image or data is converted to a second binarized image or data by a second threshold operator, the method further comprising:
determining, by the one or more processors, one or more values of features extracted from a distribution of a second power of the one or more second binarized regions, wherein the second threshold operator has a value lower than that of the threshold operator, and wherein the second power excludes power of the two or three-dimensional objects.

6. The method of claim 1, wherein the one or more features are selected from the group consisting of:
a feature associated with a time range of the one or more binarized regions identified in the spectral image or data;
a feature associated with a frequency range of the one or more binarized regions identified in the spectral image or data;
a feature associated with a time centroid of the one or more binarized regions identified in the spectral image or data;
a feature associated with a surface area of the one or more binarized regions identified in the spectral image or data;
a feature associated with a measure of eccentricity of at least one of the one or more binarized regions identified in the spectral image or data;
a feature associated with a measure of circularity of the at least one of the one or more binarized regions identified in the spectral image or data;
a feature associated with a binarized regions extent identified in the spectral image; or data
a feature associated with an orientation of an ellipse identified in the spectral image or data; and
a feature associated with a power centroid identified in the spectral image or data.

7. The method of claim 2, wherein the wavelet-based model is based on a photoplethysmographic signal.

8. The method of claim 2, wherein the wavelet-based model is based on a velocity-plethysmographic signal derived from a photoplethysmographic signal.

9. The method of claim 2, wherein the wavelet-based model is based on a cardiac/biopotential signal.

10. The method of claim 1, wherein determining the values of the wavelet-based features or parameters comprises:
determining, by the one or more processors, a wavelet-based model of a plurality of pre-defined portions within identified periodic cycles of a cardiac signal of the biophysical signal data set, wherein each of the plurality of pre-defined portions comprises an isolated cardiac waveform associated with atrial depolarization, ventricular depolarization, or ventricular repolarization; and
determining, by the one or more processors, one or more values of features extracted from high-energy components of the wavelet-based model.

11. A method of claim 1, wherein the one or more features are selected from the group consisting of:
a feature associated with a statistical assessment of a plurality of power spectral density values determined within the wavelet-based model comprising a plurality of isolated cardiac waveform associated with atrial depolarization;
a feature associated with a statistical assessment of a plurality of power spectral density values determined within the wavelet-based model comprising a plurality of isolated cardiac waveform associated with ventricular depolarization; and
a feature associated with a statistical assessment of a plurality of power spectral density values determined within the wavelet-based model comprising a plurality of isolated cardiac waveform associated with ventricular repolarization.

12. A method of claim 1, wherein the one or more features include a feature associated with an assessment of deviations from linearity in a quantile-quantile probability assessed between (i) a power spectral density values determined within the wavelet-based models and (ii) a base power spectral density function.

13. A method of claim 1, wherein the one or more features include a feature associated with an assessment in a quantile-quantile probability assessed between (i) a cumulative density distribution (CCD) values determined within the wavelet-based models and (ii) a cumulative density distribution function.

14. A method of claim 1, wherein the one or more features include a feature associated with an assessment of a kernel density estimator (KDE) fitted to a probability density distribution (PDD) function of the power spectral density function (PSD).

15. The method of claim 1 further comprising:
causing, by the one or more processors, generation of a visualization of the estimated value for the presence of the disease state or abnormal condition, wherein the generated visualization is rendered and displayed at a display of a computing device and/or presented in a report.

16. The method of claim 1, wherein the values of one or more wavelet associated properties are used in the model selected from the group consisting of a linear model, a decision tree model, a random forest model, a support vector machine model, a neural network model.

17. The method of claim 1, wherein the model further includes features selected from the group consisting of:
one or more depolarization or repolarization wave propagation associated features;
one or more depolarization wave propagation deviation associated features;
one or more cycle variability associated features;
one or more dynamical system associated features;
one or more cardiac waveform topologic and variations associated features;
one or more PPG waveform topologic and variations associated features;
one or more cardiac or PPG signal power spectral density associated features;
one or more cardiac or PPG signal visual associated features; and
one or more predictability features.

18. The method of claim 1, wherein the disease state or abnormal condition is selected from the group consisting of coronary artery disease, pulmonary hypertension, pulmonary arterial hypertension, pulmonary hypertension due to left heart disease, rare disorders that lead to pulmonary hypertension, left ventricular heart failure or left-sided heart failure, right ventricular heart failure or right-sided heart failure, systolic heart failure, diastolic heart failure, ischemic heart disease, and arrhythmia.

19. A system comprising:
a processor; and
a memory having instructions stored thereon, wherein execution of the instructions by the processor causes the processor to:
acquire a biophysical-signal data set of a subject comprising one or more first biophysical signals;
determine values of wavelet-based features or parameters that characterize properties or geometric shapes of a binarized data object generated from a wavelet transform of the biophysical-signal data set;
determine an estimated value for a presence of the disease state or abnormal condition-based, in part, on the determined values of the one or more wavelet associated properties, wherein the estimated value for the of the disease state or abnormal condition is used in a model to non-invasively estimate the presence of an expected disease state or condition,
wherein the estimated value is subsequently outputted for use in a diagnosis of the expected disease state or condition or to direct treatment of the expected disease state or condition.

20. A non-transitory computer-readable medium having instructions stored thereon, wherein execution of the instructions by a processor causes the processor to:
acquire a biophysical-signal data set of a subject comprising one or more first biophysical signals;
determine values of wavelet-based features or parameters that characterize properties or geometric shapes of a binarized data object generated from a wavelet transform of the biophysical-signal data set;
determine an estimated value for a presence of the disease state or abnormal condition-based, in part, on the determined values of the one or more wavelet associated properties, wherein the estimated value for the of the disease state or abnormal condition is used in a model to non-invasively estimate the presence of an expected disease state or condition,
wherein the estimated value is subsequently outputted for use in a diagnosis of the expected disease state or condition or to direct treatment of the expected disease state or condition.
